(19) United States
(12) Patent Application Publication
(10) Pub. No.: US 2010/0016367 A1
(43) Pub. Date: Jan. 21, 2010

(54) BIS-PYRINIDIUM COMPOUNDS

(75) Inventors: Alfred Werner Widmer, New South Wales (AU); Katrina Anne Jolliffe, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The University of Sydney, Sydney, New South Wales (AU)

(21) Appl. No.: 12/299,459

(22) PCT Filed: May 4, 2007
(86) PCT No.: PCT/AU07/00594

§ 371 Date: Feb. 10, 2009

(30) Foreign Application Priority Data
May 5, 2006 (AU) 2006902367

(51) Int. Cl. A61K 31/444; A61P 31/04; A61P 31/10; A61P 31/12; A61P 33/00; C07D 401/06

(52) U.S. Cl. 514/332; 546/255

(57) ABSTRACT

A method of treating, inhibiting, or preventing an infection in a subject is described. The method comprises administering to the subject an effective amount of at least one bis-pyridinium compound. The bis-pyridinium compound comprises two aromatic ring structures. Each of the ring structures comprises a pyridine ring, and the ring structures are linked by a linker group of at least 8 atoms in length, said linker group being attached to the nitrogen atoms of the pyridine rings. At least one substituent on at least one of the ring structures is an alkyl group having at least 2 carbon atoms, and no substituent on either of the ring structures is —OH, —SH or an amine group.

### TECHNICAL FIELD

The present invention relates to bis-pyridinium compounds which may be used for antimicrobial or antifungal applications.

### BACKGROUND OF THE INVENTION

There is a world-wide need for agents to prevent, control and treat infections. The reasons for this include development of resistance, emergence of new infective organisms and new challenges in terms of environmental contamination. There is a need for new infection control agents in the agrochemical industry, the preservation industry and elsewhere.

Some biscationic compounds are known to have antimicrobial (e.g. bactericidal or antimalarial) activity. Octenidine [a bis(alkylaminopyridinium)] is a well known disinfectant, while dequalinium [a bis(aminoquinolinium)] is used as a topical antifungal. The structures of these compounds are shown below.

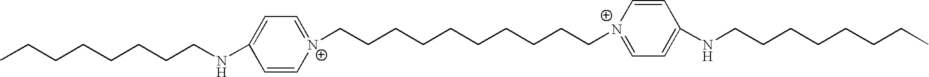

Octenidine disinfectant: strongly hemolytic and strong inhibitor of ppPLA₂.

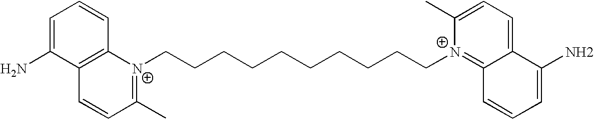

Dequalinium: topical antifungal, not haemolytic, does not inhibit PLB nor ppPLA2.

Some bis-(aminopyridinum) compounds have been investigated as antimalarials (WO9804252A1; WO9611910A1), and as antivirals, antiparasitics and antifungals (WO9805644A1).

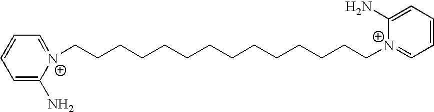

### Structure of an Antimalarial Bis(aminopyridinium)

All of the above classes of compound contain an amino substituent (primary, secondary or tertiary) attached directly to the pyridine ring(s) or to a ring fused with the pyridine ring(s).

“Gemini” or “bola” surfactants, having relatively short linker groups connecting two pyridine rings via their carbon atoms and long chains off the pyridine nitrogen atoms, have also been investigated. These are known to be disinfectants, but the long alkyl chains result in high hemolytic activity, preventing use as therapeutics. The linkers between the two pyridine groups are generally from 4-8 atoms in length and may contain amides, ethers, thioethers and esters. The linker may be attached directly to the rings or may be attached via S, O—, CO, COO—, CONH. These compounds have also been used as transfection agents.

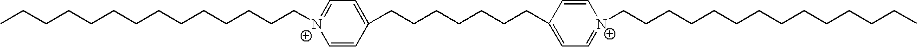

### Structure of a Gemini Surfactant

Bis(alkylpyridiniums), in which the linker joins the ring nitrogen atoms of two pyridines and at least one alkyl chain is attached to at least one carbon atom of each pyridine ring, have been found to act as neuronal nicotinic acceptor antagonists (WO2005066129 A2) and their use has also been described in the preparation of photographic materials with high green-sensitivity (U.S. Pat. No. 4,554,628 and U.S. Pat. No. 4,552,837).

### OBJECT OF THE INVENTION

It is an object of the present invention to overcome or substantially ameliorate at least one of the above disadvantages, or at least to provide alternatives to the methods known in the prior art for treating infections.

### SUMMARY OF THE INVENTION

In a first aspect of the invention there is provided a method of treating, inhibiting, or preventing an infection in a subject, said method comprising administering to said subject an effective amount of a bis-pyridinium compound, wherein said bis-pyridinium compound comprises two aromatic ring structures and wherein:
each of the ring structures comprises a pyridine ring,
the ring structures are linked by a linker group of at least 8 atoms (e.g. at least 8 carbon atoms) in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group.
   Each of the ring structures may, independently, be a pyridine ring or a fused pyridine ring (i.e. a pyridine ring fused with at least one other aromatic ring) e.g. a quinoline, isoquinoline or acridine ring. Each of the ring structures may, independently, be monocyclic, bicyclic, tricyclic or polycyclic.

The bis-pyridinium compound may consist of two aromatic ring structures wherein:
each of the ring structures comprises a pyridine ring,
the ring structures are linked by a linker group of at least 8 atoms (e.g. at least 8 carbon atoms) in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group.

The infection may be a microbial infection, a bacterial infection, a fungal infection, an amoebic infection, a viral infection, a parasitic infection, a mould infection or a helminthic infection or some other type of infection. The infection may be an infestation. The bis-pyridinium compound may be administered to the subject either topically (e.g. in the form of a cream, salve, lotion, ointment, balm, spray) or systemically (e.g. by injection, ingestion, inhalation or some other systemic route).

The bis-pyridinium compound may be a bis-pyridinium salt. It may be a halide salt or some other type of salt. The linker group may be between 8 and 18 atoms long. It may comprise a hydrocarbon chain, or it may be a hydrocarbon chain. The main chain of the linker group may be, or may comprise, a hydrocarbon chain, optionally substituted. The bis-pyridinium compound may be such that no substituent on either ring structure, other than the linker group, has more than 10 carbon atoms in a straight chain. The two ring structures may be the same or different. The substitution on the two ring structures may be the same or it may be different. Each ring may have one or more (e.g. 2, 3, 4 or 5) substituents. Each substituent may be an alkyl substituent. Each substituent may optionally and independently comprise, or be, one or more ether, ester, amide or carbonyl groups. Each substituent may, independently, be optionally substituted, e.g. by halogen, hydroxyl, thiol, amine, aryl and/or other groups. Each substituent may, independently, be straight chain, branched and/or cyclic. Each substituent on the ring structures may, independently, have between 0 and 10 carbon atoms in a straight chain, i.e. 0, 1, 2, 3, 4, 5, 6, 7, 8, 9 or 10 carbon atoms (or between 0 and 5, 5 and 10, 1 and 10, 1 and 5, 2 and 8 or 3 and 7), provided that at least one of the substituents has at least 2 carbon atoms. Any one or more of the substituents may have more than 10 carbon atoms, provided that there are no more than 10 carbon atoms in a straight chain in the substituent. Thus for example a substituent may be a 6-phenylhexyl group, or a 2,2,3,3-tetraethylpentyl group, both of which have more than 10 carbon atoms, but neither of which have more than 10 carbon atoms in a straight chain. Each substituent on the ring structures may, independently, have between 0 and 10 carbon atoms, i.e. 0, 1, 2, 3, 4, 5, 6, 7, 8, 9 or 10 carbon atoms (or between 0 and 5, 5 and 10, 1 and 10, 1 and 5, 2 and 8 or 3 and 7), provided that at least one of the substituents has at least 2 carbon atoms. The bis-pyridinium compound may, optionally, be such that it does not comprise an —OH, —SH, —NH₂ or —NHR group (where R is alkyl or aryl). The substituents may for example be alkyl groups (linear, branched or cyclic), nitrile groups, nitro groups, halides (chloride, bromide, iodide), aromatic groups, arylalkyl groups or hydrogen.

The bis-pyridinium compound may have an MIC (mean inhibitory concentration) against a microorganism, e.g. C. neof. ATCC 90112 or C. albicans ATCC 10231, of less than about 11 micromolar. It may have an MIC against said organism of less than about 10 micrograms per millilitre

In an embodiment, the bis-pyridinium compound has structure I,

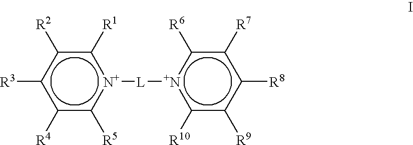

wherein
at least one of R¹ to R¹⁰ is an alkyl group having at least 2 carbon atoms,
none of R¹ to R¹⁰ is —OH, —SH or an amine group, and
L is a linker group which is at least 8 atoms in length.
   The bispyridinium compound may be such that none of R¹ to R¹⁰ has more than 10 carbon atoms in a straight chain.

The method may comprise administering a mixture of bis-pyridinium compounds, more than one of which (optionally all of which) are as described in the first aspect above.

In a second aspect of the invention there is provided a method of killing an organism comprising exposing said organism to an effective amount of at least one bis-pyridinium compound, wherein said bis-pyridinium compound is as described in the first aspect (including the options and embodiments therein). The exposing may comprise administering the at least one bis-pyridinium compound to a subject, said subject being infected, or infested, by the organism. The exposing may comprise disinfecting a locus by contacting the locus and optionally its surrounds with an effective disinfecting amount of the at least one bis-pyridinium compound. Alternatively the exposing may comprise disinfecting a surface by contacting the surface with a disinfecting amount of the at least one bis-pyridinium compound. The organism may be a microorganism. The organism may be for example a bacterium, a fungus, an amoeba, a virus, a helminth, a parasite or some other type of organism. The effective amount may be a lethal amount for said organism.

The invention also provides a method of inhibiting or preventing growth of an organism, e.g. a fungus, comprising exposing said organism to an effective amount of at least one bis-pyridinium compound, wherein said bis-pyridinium compound is as described in the first aspect. The effective amount may be a fungistatically effective amount.

In a third aspect of the invention there is provided a biocidal or fungistatic bis-pyridinium compound comprising two aromatic ring structures, wherein:
each of the ring structures comprises a pyridine ring,
the ring structures are linked by a linker group of at least 8 atoms (e.g. at least 8 carbon atoms) in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group.

Each of the ring structures may, independently, be a pyridine ring or a fused pyridine ring (i.e. a pyridine ring fused with at least one other aromatic ring) e.g. a quinoline, isoquinoline or acridine ring. The bis-pyridinium compound may be microbiocidal, bactericidal, fungicidal or antiviral. The compound may be as described in the first aspect (including the options and embodiments therein).

In a fourth aspect of the invention there is provided the use of a bis-pyridinium compound as described in the first aspect (including the options and embodiments therein) in the manufacture of a medicament for the treatment of an infection. The infection may be a microbial infection, a bacterial infection, a fungal infection, an amoebic infection, a viral infection, a parasitic infection or infestation or some other type of infection or infestation. The infection may be in and/or on a subject such as a mammal (e.g. an animal or a human) or other animal or a plant.

In a fifth aspect of the invention there is provided a biocidal formulation comprising a bis-pyridinium compound as described in the first aspect (including the options and embodiments therein) together with one or more acceptable adjuvants and/or carriers. The formulation may be a solution, a suspension, an emulsion, a dispersion. It may be a liquid formulation. It may be a cream or a paste or a powder. It may be for example in the form of a cream, salve, lotion, ointment, balm or a spray. The formulation may be a medicament, a pharmaceutical preparation, an agricultural preparation, a veterinary preparation, a disinfectant or some other type of biocidal formulation. It may be a preparation that is pharmaceutically, veterinarily or agriculturally acceptable.

In a sixth aspect of the invention there is provided a process for making a biocidal bis-pyridinium compound as described in the first aspect (including the options and embodiments therein) comprising reacting one or more pyridine compounds with a linker reagent, said linker reagent having two leaving groups joined by a linker group and said linker group being more than 8 atoms in length. Each of the one or more pyridine compounds may have an alkyl substituent on a ring carbon, said alkyl substituent having at least 2 carbon atoms, and none of the one or more pyridine compounds may have a substituent that is an —OH, —SH or an amine group, i.e. the pyridine compound(s) does not have a —OH, —SH or an amine group directly attached to the pyridine ring. The pyridine compound(s) may optionally have one or more substituents that are substituted by a —OH, —SH or an amine group, e.g. the pyridine compound(s) may have a hydroxypropyl substituent. The or each pyridine compound may, independently, be a pyridine compound or a fused pyridine compound (i.e. a pyridine ring fused with at least one other aromatic ring) e.g. a quinoline, isoquinoline or acridine compound. The or each pyridine compound may, independently, be monocyclic, bicyclic, tricyclic or polycyclic.

In one embodiment a single pyridine compound is used in the process. The linker reagent, and/or the linker group, may be symmetrical or may be asymmetrical. The bis-pyridinium compound may be a symmetrical bis-pyridinium compound (i.e. the substituents on the two ring structures may be the same and the two ring structures may be the same).

In another embodiment the bis-pyridinium compound is an asymmetrical bis-pyridinium compound (i.e. the substituents on the two ring structures are not the same or the two ring structures are not the same), and a mixture of pyridine compounds is used in the process. The linker reagent, and/or the linker group, may be symmetrical or may be asymmetrical. In this embodiment, a mixture of bis-pyridinium compounds may initially be produced. The process may additionally comprise separating the desired asymmetrical bis-pyridinium compound. The separating may use one or more known separation methods, e.g. chromatography, recrystallisation, fractional crystallisation etc.

In a seventh aspect of the invention there is provided a biocidal bis-pyridinium compound made by the process of the sixth aspect (including the options and embodiments therein).

In an eighth aspect of the invention there is provided the use of a bis-pyridinium compound as a disinfectant and/or as a biocide, said bis-pyridinium compound being as described in the first aspect (including the options and embodiments therein).

In a ninth aspect of the invention there is provided the use of a bis-pyridinium compound as a phospholipase inhibitor, said bis-pyridinium compound being as described in the first aspect (including the options and embodiments therein).

In a tenth aspect of the invention there is provided a bis-pyridinium compound, wherein said bis-pyridinium compound comprises two aromatic ring structures and wherein:
each of the ring structures comprises a pyridine ring,
the ring structures are linked by a linker group of at least 8 atoms (e.g. at least 8 carbon atoms) in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group,
   when used in an effective amount to treat, inhibit, or prevent an infection in a subject or to disinfect an object, locus and/or its surrounds or a surface. The bis-pyridinium compound may be as described in the first aspect of the invention, including the options and embodiments therein.

### DETAILED DESCRIPTION OF THE PREFERRED EMBODIMENTS

The present invention discloses bis-pyridinium compounds, in particular bis(alkylpyridinium)compounds, more particularly bis(alkylpyridinium)alkanes, in which the pyridine rings are linked through the nitrogen atoms of the pyridine rings. These compounds may be used as antimicrobial agents. They may be not strongly haemolytic. They may be not haemolytic. The bis-pyridinium compounds do not bear amino substituents on the pyridine rings. Members of this class of bis-cationic compounds offer broad-spectrum antifungal activity, are selective for the fungal phospholipase enzyme target, and may have MICs below about 11 micromolar or as low as 1 micromolar or less, or below about 10 microgram/ml or below about 1 microgram/ml. This class offers a simple, novel, structural space for antimicrobial compounds. The compounds described herein are novel as antimicrobials e.g. antibacterials and/or antifungals. They may be antiviral compounds. They are novel as phospholipase inhibitors. Certain related compounds are known, however the more potent antifungal compounds are hitherto unknown. Table 1 lists in vitro antifungal activity for a range of bis-(alkylpyridinium)alkane salts. For comparision of activity, Table 2 lists in vitro antifungal activity of 1,12-bis(4-pentylpyridinium)dodecane, a representative bis-(alkylpyridinium)alkane salt, in comparison to Amphotericin B and tables 1a, 1b and 2a provide other related experimental data.

Phospholipases are known to be present in all microbes and parasites and consequently phospholipase inhibitors may be broadly antimicrobial. The present inventors have found that bis-alkylpyridinium compounds are effective phospholipase inhibitors and consequently may provide improved selectivity, potency, utility (topical, systemic), stability, availability, metabolism, toxicity, etc. when used in antimicrobial applications. Table 3 shows the degree of inhibition of Secretory Ciyptococcal H99 Phospholipase B (PLB) and ppPLA₂ activites by three representative bis(alkylpyridinium)alkanes.

The synthesis of bis-alkylpyridinium compounds is simple, and is based on relatively inexpensive starting materials. The compounds are comparatively stable to enzymatic and thermal degradation. Additionally, the compounds are commonly non-haemolytic, which renders them suitable for administration to a subject by ingestion. The compounds may have sufficiently low haemolytic activity to render them suitable for administration to a subject by ingestion. The compounds may be effective as broad spectrum antiinfective compounds. They may be active against parasites and/or helminths (e.g. nematodes). They may be active against yeasts. They may be active against moulds. They may be active against dermatophytes. They may be active against fungi. They may be used topically and/or systemically. They may be administered topically, enterally or parenterally. They may be used for control of parasites, microbial infections, mould infections, fungal infections, viral infections and/or nematodes in and/or on a subject. They may be useful in control of skin and/or toenail infections.

The subject may be an animal, e.g. a human or a non-human mammal or some other vertebrate. The vertebrate may be a mammal, a marsupial, a fish, a bird or a reptile. The mammal may be a primate or non-human primate or other non-human mammal. The mammal may be selected from the group consisting of human, non-human primate, equine, murine, bovine, leporine, ovine, caprine, feline and canine. The mammal may be selected from a human, horse, cattle, sheep, dog, cat, goat, llama, rabbit and a camel, for example. Alternatively the subject may be a plant. The plant may be a tree, a shrub, a bush, a crop, a cereal etc. for example barley or canola (rapeseed).

Preferred compounds include biocidal bis-pyridinium compounds comprising two pyridine rings, and a linker group which joins the ring nitrogen atoms of the two pyridine rings. It will be understood that in the present specification, when referring to a pyridine ring of a bis-pyridinium compound, the ring is positively charged, and is therefore a pyridinium ring.

The ring structures of the bis-pyridinium compounds of the present invention may be the same or may be different. Thus the bis-pyridinium compound may be symmetrical or asymmetrical. The pyridine rings of these compounds may each, independently, be fused (e.g. part of a quinoline, isoquinoline, benzoquinoline or acridine group) or may be unfused. The ring structures may be substituted, for example by a halogen (e.g. fluorine, chlorine, bromine or iodine) and/or a nitrile. The ring structures are not directly substituted by —OH, —SH, —NH₂, NHR or NRR^{a}. The ring structures may be such that they are not directly substituted by an acidic group. The ring structures may also be such that they are not directly substituted by —COOH and/or —SO₂H and/or —SOH and/or —PO₃H and/or some other acidic species. In some embodiments, one or more of —OH, —SH, —NH₂, NHR and NRR^{a}, and optionally —COOH, —SO₂H, —SOH or PO₃H, or some other acidic species, may be present provided that they are not directly attached to either of the ring structures. They may or may not be not substituted (i.e. may be unsubstituted) by COOR or CONHR (where R and R^{a} are, independently, alkyl or aryl groups). One or both of the ring structures has an alkyl substituent, and may, independently, have more than one alkyl substituent (e.g. 2, 3, 4 or 5 alkyl substituents). At least one of the alkyl substituents has at least 2 carbon atoms. The bis-pyridinium compound may be such that no substituent on the ring structures other than the linker group has more than 10 carbon atoms. At least one alkyl substituent on at least one of the ring structures has between 2 and 10 carbon atoms (inclusive), or between 4 and 10, 2 and 8, 2 and 6, 4 and 8, 4 and 6, 6 and 10, 8 and 10 or 6 and 8 carbon atoms, and may have 2, 3, 4, 5, 6, 7, 8, 9 or 10 carbon atoms. The bis-pyridinium compound may be such that no substituent on the ring structures other than the linker group has more than 10 carbon atoms in a straight chain. The alkyl groups may, independently, be branched, straight chain or cyclic. They may optionally be substituted, e.g. by a phenyl group, a halogen (e.g. chloride, fluoride, bromide, iodide) and/or a nitrile. They may be unsaturated (i.e. may be alkenyl or alkynyl groups). They may optionally have no substituent having an active hydrogen atom. They may, optionally, have no substituent which is —OH, —SH, NH₂, NHR or NRR^{a} where R and R^{a} are, independently, alkyl or aryl groups, although in some embodiments such groups may be present when attached to a substituent on one or both of the ring structures.

The bis-pyridinium compound may have any suitable counterion. The counterion may have a −1 or a −2 charge or may have a negative charge of more than 2. It may be monovalent, divalent, trivalent, tetravalent or polyvalent. It may be an inorganic or an organic counterion. It may be a monomeric counterion, an oligomeric counterion or a polymeric counterion. It may for example be fluoride, chloride, bromide, iodide, acetate, propionate, trifluoroacetate, hexafluorophosphate, carbonate, sulfate, adipate, alginate, ascorbate, aspartate, benzenesulfonate, benzoate, borate, butyrate, camphorate, camphorsulfonate, citrate, digluconate, cyclopentanepropionate, dodecylsulfate, dodecylbenzenesulfonate, ethanesulfonate, famarate, glucoheptonate, glycerophosphate, hemisulfate, heptonate, hexanoate, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, stearate, succinate, tartrate, thiocyanate, toluenesulfonate, undecanoate, valerate or some other suitable counterion. The counterion may have buffering capability, e.g. may be bicarbonate, bisulfate, hydrogen phosphate, dihydrogen phosphate etc.

The bispyridinium compounds of the present invention may have an MIC against a target organism of about 11 micromolar or less, or less than about 10, 9, 8, 7, 6, 5, 4, 3, 2 or 1 micromolar, for example between about 0.01 and 10, 0.01 and 9, 0.01 and 8, 0.01 and 7, 0.01 and 6, 0.01 and 5, 0.01 and 4, 0.01 and 3, 0.01 and 2, 0.01 and 1, 0.01 and 0.5, 0.01 and 0.1, 0.01 and 0.05, 1 and 10, 1 and 5, 1 and 2, 0.05 and 2, 0.1 and 11, 0.1 and 10, 0.1 and 9, 0.1 and 8, 0.1 and 7, 0.1 and 6, 0.1 and 5, 0.1 and 4, 0.1 and 3, 0.1 and 2 or 0.1 and 1 micromolar, e.g. about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5,3, 3.5, 4, 4.5, 5, 5.6, 6, 6.5, 7, 7.5, 8, 8.5, 9, 9.5, 10, 10.5 or 11 micromolar. In some embodiments, the MIC may be less than about 20 micromolar, or less than about 19, 18, 17, 16, 15, 14, 13 or 12 micromolar, or between about 10 and about 20, about 10 and 15 or about 15 and 20 micromolar, e.g. about 12, 13, 14, 15, 16, 17, 18, 19 or 20 micromolar. It may have an MIC against the target organism of about 10 micrograms/ml or less, or less than about 9, 8, 7, 6, 5, 4, 3, 2 or 1 micrograms/ml, for example between about, 0.01 and 10, 0.01 and 9, 0.01 and 8, 0.01 and 7, 0.01 and 6, 0.01 and 5, 0.01 and 4, 0.01 and 3, 0.01 and 2, 0.01 and 1, 0.01 and 0.5, 0.01 and 0.1, 0.01 and 0.05, 1 and 10, 1 and 5, 1 and 2, 0.05 and 2, 0.1 and 10, 0.1 and 9, 0.1 and 8, 0.1 and 7, 0.1 and 6, 0.1 and 5, 0.1 and 4, 0.1 and 3, 0.1 and 2 or 0.1 and 1 micrograms/ml, e.g. about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.6, 6, 6.5, 7, 7.5, 8, 8.5, 9, 9.5. or 10 micrograms/ml. The target organism may be a yeast, a mould, a bacterium, a dermatophyte or some other organism. The target organism may be for example a Candida spp., Aspergillus spp., Scedosporium spp., Fusariun spp., Cryptococcus spp., Leptosphaeria spp., Staphlylococcus spp., Streptococcus spp., or Enterococcus spp. e.g. Cryptococcus neoformans, Cryptococcus gattii, Candida albicans, Candida krusei, Candida parapsilosis, Candida glabrata, Candida tropicalis, Candida guilliermondii, Aspergillus fumigatus, Aspergillus terreus, Aspergillus flavus, Aspergillus niger, Aspergillus nidulans, Leptosphaeria maculans (blackleg), Scedosporium prolificans, Scedosporium apiospermum, Fusarium solani, Fusarium oxysporum, Cunninghamella bertholletiae, Absidia corymbifera, Rhizopus oiyzae, Exophiala spinifera, Exophiala jeanselme, Paeciloinyces lilacinus, Trichophyton rubrum, Trichophlyton mentagrophytes, Trichophyton tonsurans, Trichophlyton soudanense, Trichophyton violaceum, Epidermoplzyton floccosum, Microsporum canis, Microsporum gypseum, Microsporum cookie, Staphlylococcus aureus, Staphylococcus epidermidis, Streptococcus pneumoniae, Enterococcus faecalis or Enterococcus faecium. The bispyridinium compound may have haemolytic activity of less than about 5%, or less than about 4, 3, 2, 1, 0.5, 0.1, 0.05 or 0.01% (for example about 0.01, 0.05, 0.1, 0.5, 1, 1.5, 2, 2.5, 3, 3.5, 4, 4.5 or 5%) at a concentration of about 15 times that of the MIC against the target organism, or at a concentration of between about 10 and 20, 10 and 15, 15 and 20 or 13 and 17 times that of the MIC against the target organism (e.g. at a concentration of about 10, 1 1, 12, 13, 14, 15, 16, 17, 18, 19 or 20 times the MIC against the target organism). A low haemolytic activity may render the compounds suitable for use internally. However compounds according to the invention which have a high haemolytic activity (relative to the activities described above) may find application where haemolysis is not a disadvantage, e.g. as disinfectants or in topical applications. Table 4 shows haemolytic activity of selected bis(alkylpyridinium)alkane salts as a function of concentration. The bispyridinium compounds described herein may be effective against a range of microorganisms. They may be effective against gram positive bacteria, or against gram negative bacteria or against both gram positive and gram negative bacteria. Table 5 shows in vitro antibacterial properties of 1,12-bis(4-pentylpyridinium)dodecane against a range of different bacteria.

The linker group of the bis-pyridinium compound is greater than 8 atoms in length, or may be greater than about 10, 12, 14 or 16 atoms in length. It may be between 8 and 18 atoms long, or between 10 and 18, 8 and 16, 8 and 14, 8 and 12, 10 and 16, 10 and 14, 10 and 12, 12 and 18, 14 and 18, or 12 and 16 atoms long, or it may be greater than 18 atoms long. It may be 8, 9, 10, 11, 12, 13, 14, 15, 16, 17 or 18 atoms long. It may comprise a hydrocarbon chain. It may comprise a straight chain or a branched chain, and may comprise an alicyclic or aromatic ring. It may or may not comprise one or more heteroatoms, e.g. ether, thioether, amide, ester or amine groups and/or linkages. If an amine is present, it should be a tertiary amine, i.e. it should be —N(R)—, where R is not H, and may be aryl or alkyl. It may be a hydrophobic linker group. Suitable linker groups include straight chain —C₁₁H₂₂—, —C₁₂H₂₄—, —C₁₄H₂₈—, —C₁₆H₃₂—, —C₁₈H₃₆—. These may optionally be substituted, for example by alkyl (e.g. C1 to C6 straight chain, C3-C10 branched or cyclic) groups or other suitable substituents. The linker may comprise one or more heteroatoms, aromatic groups or alicyclic groups. The linker may be symmetrical or asymmetrical. Thus for example the linker may comprise —C₆H₁₂OC₆H₁₂—, —C₄H₈OC₈H₁₆—, —C₇H₁₄SC₇H₁₄—, —C₈H₁₆N(Me)C₈H₁₆—, C₅H₁₀—Ph—C₅H₁₀, —C₆H₁₂-(cyclo-C₆H₁₂)—C₆H₁₂— (wherein Ph represents ortho, meta or para-phenylene, and cyclo-C₆H₁₂ represents 1,2-, 1,3- or 1,4-cyclohexylene) or other groups that will be readily appreciated by those skilled in the art. The linker group may be saturated or unsaturated, and may be multiply unsaturated (e.g. 2, 3, 4 or 5 units of unsaturation). It may comprise one or more double bonds and/or triple bonds and/or aromatic rings rings (e.g. phenyl naphthyl, anthracyl groups etc.). If more than one of these is present, they may be conjugated or unconjugated. The linker group may comprise alicyclic rings e.g. cyclohexyl, cyclopentyl, cyclobutyl, cyclopropyl, cycloheptyl, cyclohexenyl, cyclopentenyl etc. The linker may be optionally substituted, for example with a halogen (e.g. fluorine, chlorine, bromine or iodine), an alkyl group, an alkenyl group, an alkynyl group or an aromatic group. The linker group may be attached to the pyridine rings by a C—N bond.

The bispyridinium compounds may be active as biocides, antifungals, antiviral agents, antimicrobials, antiparasitics, disinfectants, antiseptics for hospital and/or environmental uses. They may be active as more than one of these. The compounds may be used in agrochemistry, hygiene/disinfectant applications, and as preservatives in wood, textiles, paints, glues, oils, animal feeds etc. The compounds comprise a novel pharmacophore which offers advantages over existing antimicrobial compounds, for example ease of synthesis, chemical stability, economy of synthesis, and/or resistance to currently used compounds.

It will be understood that the methods described herein may comprise use of a mixture of bis-pyridinium compounds, more than one (optionally all) of which are as described in the first aspect of the invention. When used in combination, each may be in an effective dose (for preventing an infection, for killing an organism etc.), or the combined concentration of the combination of bis-pyridinium compounds may be effective for the selected purpose. In the latter case one or more of the individual bis-pyridinium compounds (optionally all) may be in a concentration below the effective concentration for that compound, however the mixture may still be effective. The effects of the individual compounds in such a mixture may be additive. The effects may be synergistic.

The compounds may be used as disinfectants. They may be dissolved, suspended or emulsified in a solvent, e.g. an alcohol (methanol, ethanol, isopropanol). The solvent may be a polar organic solvent. It may be aqueous. It may be water. The compounds may be applied by spraying, wiping etc. They may be incorporated (e.g. impregnated) into disinfectant wipes.

The bispyridinium compounds described herein may be combined with one of more acceptable adjuvants and/or carriers to form a formulation. The formulation may also comprise other (non-bispyridinium) antimicrobial compounds. The formulation may comprise one or more than one (e.g. 2, 3, 4 or 5) different bis-pyridinium compounds as described in the present invention. The formulation may be a solution, a suspension, an emulsion or a dispersion. It may be a liquid formulation. It may be a cream or a paste. It may be a solid, e.g. a powder. It may be for example in the form of a cream, salve, lotion, ointment, balm or a spray. The formulation may be a medicament, a pharmaceutical preparation, an agricultural preparation, a veterinary preparation, a disinfectant or some other type of biocidal formulation. The adjuvants and/or carriers may be agriculturally, pharmaceutically or veterinarily acceptable, depending on the application. They may be non-toxic and/or non-harmful to the subject to which they are administered.

In a therapeutic application, compositions (formulations) may be administered to a patient already suffering from a disease, in an amount sufficient to cure or at least partially arrest the disease and its complications. The composition should provide a quantity of the compound or agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular), oral or topical route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysiloxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethylcellulose, polyvinylpyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations of the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous medium, optionally including a surface active agent. Suitable solvents for the preparation of an oily solution include glycerol, diluted alcohol and propylene glycol.

Lotions according to the present invention include those suitable for application to the skin or eye. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

### Examples

### Biological Methods

### Materials

All chemical reagents were obtained from the Sigma Chemical Co. unless otherwise specified and were of the highest purity available. General laboratory chemicals were purchased from AJAX (Australia) unless otherwise stated. Radiochemicals were supplied by Amersham Life Sciences unless otherwise specified. Solvents of analytical grade were obtained from the Sigma Chemical Co.

### Fungal Isolates, Media and Inoculum

A virulent clinical isolate of C. neoformans var. grubii (serotype A), H99, that produces high levels of secreted phospholipase B activity was used for the mouse model and studies of inhibition of phospholipase activities. For fungicidal activity, C. neoformans ATCC 90112 was used. Isolate H99 was supplied by Dr Gary Cox (Duke University Medical Center, Durham, N.C., USA), and subcultured onto Sabouraud dextrose agar (SDA) at 30° C. C. neoformans inoculates used in the mouse model and antifungal susceptibility assay were prepared by transferring a loop of H99 or ATCC 90112 into a tube with 25 mL of yeast nitrogen broth (YNB) and left overnight on a shaking incubator at 35° C. The tube was centrifuged for 10 mins at 12,000 rpm in a Beckman TJ-6 centrifuge and the supernatant discarded. The pellet was washed twice with 25 mL of saline and centrifuged at 12,000 rpm for 5 mins. Finally, the cell pellet was resuspended in 10 mL of saline and the cells counted.

### Preparation of Supernatants Containing Secreted Phospholipase Activities

Isolate H99 was grown to confluence on SDA in 16 cm diameter Petri dishes for 48 h at 30° C. in air. Cells scraped from 10-20 dishes were washed twice with isotonic saline (50 mL and 20 mL) and once with 20 mL of imidazole buffer (10 mM imidazole, 2 mM CaCl₂, 2 mM MgCl₂, 56 mM D-Glucose, made up in isotonic saline, pH 5.5) by centrifugation at 2,800 rpm for 30 mins in a Beckman TJ-6 centrifuge. The pellet was resuspended in a volume of this buffer of about 10% of the cell volume, and incubated for 24 h at 37° C. The cell-free supernatant was separated by centrifugation at 1,400 rpm for 10 mins and stored in aliquots of 100 μL at −70° C.

### Enzyme Activity and Inhibition Assays

A published radiometric assay method for determining phospholipase activities was used (Chen et al., 1997) with a final volume of 125 μL, using 125 mM imidazole acetate buffer (assay buffer, pH 4.0, 5 mM EDTA) at 37° C. For the determination of secreted PLB activity, carrier dipalmitoylphosphatidylcholine (DPPC, final concentration 800 μM, in CHCl₃) and 1,2-di[1-¹⁴C]-palmitoylphosphatidylcholine (20,000 dpm) were dried under nitrogen and suspended in 125 mM imidazole acetate buffer (pH 4.0) by sonication using a Branson 450 sonifier. The reaction time was 22 mins using 1 μg total protein. PLB activity was determined by the rate of decrease of the radiolabelled PC substrate, with appearance of the label in the free fatty acid.

LPL and LPTA activities were measured simultaneously in a reaction mixture containing 1-[¹⁴C]-palmitoyl-lyso-PC (25,000 dpm) and carrier palmitoyl-lyso-PC (final concentration 200 μM) in assay buffer. The reaction time was 15 s with (1 μg) of total protein and LPL activity was measured by the rate of loss of 1-[¹⁴C]-palmitoyl lyso-PC with release of radiolabelled fatty acids. LPTA activity was estimated from the rate of formation of radiolabelled DPPC. All reactions were terminated by adding 0.5 mL of 2:1 chloroform/methanol. Reaction products were extracted by the method of Bligh and Dyer (Bligh et al., 1959), separated by TLC and quantified as previously described (Chen et al., 1997). Inhibition assays were performed in triplicate. The concentrations of test compounds in the assays were 250, 25 and 2.5 μM. The same assay was used to detect PLA activity and inhibition.

### Haemolytic Activity Assay

Human blood was collected in 10 mL Vacutainer tubes containing potassium-EDTA as anticoagulant. The blood from each Vacutainer was transferred to a 50 mL centrifuge tube and the cells washed three times with 30 mL of calcium- and magnesium-free phosphate-buffered saline (PBS; Gibco). Cells were collected by centrifugation at 2,000×g for 10 mins in a Beckman TJ-6 centrifuge. The third supernatant was clear and colourless. Cells were stored in PBS (20 mL) for up to two weeks. Then 0.5 mL cell suspension in PBS was mixed with (0.5 mL) of test substance using stock solutions of concentrations 700, 350, 175, 70 and 7 μM (final erythrocyte concentration around 0.5×10⁹ per mL). The mixtures were incubated at 37° C. for 1 h with gentle shaking, centrifuged at 2,000×g for 10 mins, the supernatant diluted 10 fold with PBS, and optical density measured at 540 nm. The values for 0% and 100% lysis were determined by incubating cells with PBS or 0.1% (w/v) Triton X-100 (in water), respectively. Assays were carried out in triplicate. The concentrations of test compounds in the assays were 350, 175, 87.5, 35 and 3.5 μM.

### Antifungal Susceptibility Assay

Antifungal activity was measured by a standard microdilution method (Ghannoum et al, 1992). The minimal inhibitory concentration (MIC) of each compound was defined as that which produced no visible growth after 48 h of culture (Candida; 1-5×10² CFU/well) or 72 h (Cryptococcus; 1-5×10³ CFU/well) at 37° C. The fungal strains tested were C. neoformans ATCC 90112 and C. albicans ATCC 10231. These were prepared as described above (Fungal isolates, media and inoculum). The concentrations of test compounds (made from 700 μM stock solution in PBS) in wells 2 to 11 were 350, 175, 87.5, 44, 22, 11, 5.5, 2.7, 1.4 and 0.7 μM. All tests were performed in duplicate.

### Chemistry

### General Methods

Melting points were determined using a Gallenkamp melting point apparatus and are uncorrected. Infrared absorption spectra were obtained using a Shimadzu FTIR—8400S spectrometer as a thin film between sodium chloride plates. ¹H nuclear magnetic resonance spectra were recorded using a Bruker Avance DPX 200 at a frequency of 200.13 MHz and chemical shifts are reported as parts per million (ppm) with deuterochloroform (CDCl₃; δ_{H} 7.26) or deuteromethanol (MeOD; δ_{H} 3.31) as internal reference. The data are reported as chemical shift (δ), multiplicity (s=singlet, d=doublet, t=triplet, q=quartet, m=multiplet), coupling constant (J Hz), relative integral and assignment. ¹³C nuclear magnetic resonance spectra were recorded using a Bruker Avance DPX 200 spectrometer at a frequency of 50.3 MHz and chemical shifts are reported as parts per million (ppm) downfield shifts with deuteromethanol (δ 49.0) as internal reference. High and low resolution mass spectra were recorded using positive electroscopy ionization on a Finnigan LCQ or a Finnigan MAT 900 XL ion trap mass spectrometer (ESI). Analytical thin layer chromatography (TLC) was performed using precoated silica gel plates (Merck Kieselgel 60 F254) and visualized using a basic KMnO₄ staining reagent. Preparative column chromatography was carried out using Merck Kieselgel 60 silica gel (SiO₂, 0.04-0.065 mm) with the indicated solvent systems. Ratios of solvents for TLC and column chromatography are expressed in (v/v) as specified. All solvents were distilled before use.

### Synthesis of Pyridines

A general process for making these compounds may be found in: J. A. Joule and G. F. Smith, Heterocyclic Chemistry, 2nd Edn., 1978, Van Nostrand Reinhold Company Ltd., London, the contents of which are incorporated herein by cross-reference.

### 4-Butylpyridine

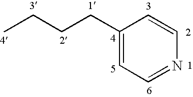

4-Picoline (3.00 g, 32.00 mmol) was dissolved in (14 ml) of dry THF and the solution was cooled to −78° C. with an acetone-dry ice bath. n-Butyllithium (16.80 ml, 42.00 mmol) was added slowly while keeping the internal ° T below −50° C. The reaction was allowed to warm up to room ° T and then stirred at 40 to 45° C. for 2 h. THF (14 ml) was added to dissolve the 4-picolyllithium slurry to give a deep orange solution. The solution was cooled down to 0° C. and carefully added into the solution of 1-bromopropane (5.02 ml, 64.00 mmol) in THF (6.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6× size of column) and the solvent was evaporated by water aspirator. The residue was further purified by flash chromatography (Hex/EtOAc 1.5/1) and the combined fractions were concentrated by water aspirator to give the above compound as a light yellow liquid (0.73 g, 20%). ¹H NMR (200 MHz, CDCl₃): δ 8.46 (2H, d, J=6.5 Hz, CH (2,6)), 7.09 (2H, d, J=6.5 Hz, CH(3,5)), 2.59 (2H, t, J=7.6 Hz, CH₂(1′)), 1.64 (2H, m, CH₂(2′)), 1.31 (2H, m, CH₂(3′)), 0.91 (3H, t, J=7.6 Hz, CH₃(4′)).

### 3-Pentylpyridine

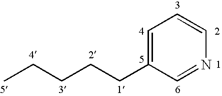

A solution of n-butyllithium (4.73 ml, 11.81 mmol) was added to a solution of diisopropylamine (1.66 ml, 11.81 mmol) in dry THF (60 ml) at −15° C. After stirring for 30 min, 3-picoline (1.00 g, 10.74 mmol) was added dropwise. The resulting red solution was stirred for 1 h at −15° C. and then a solution of 1-bromobutane (1.22 ml, 11.28 mmol) in dry THF (5.5 ml) was added in one portion. The reaction was allowed to gradually warm up to room ° T and stirred overnight. Et₂O was added and the reaction mixture washed with 1M NH₄Cl solution (3×50 ml) dried with Na₂SO₄ and evaporated to dryness with the water aspirator. The residue was further purified by flash chromatography (Hex/EtOAc 1.5/1) and the combined fractions were concentrated by water aspirator to give 2 as a light yellow liquid (0.68 g, 46%). ¹H NMR (200 MHz, CDCl₃): δ 8.43 (2H, s, CH (2,6)), 7.51 (1H, m, CH(4)), 7.24 (1H, m, CH(3)), 2.60 (2H, t, J=7.6 Hz, CH₂(1′)), 1.64 (2H, m, CH₂(2′)), 1.32 (4H, m, CH₂(3′,4′)), 0.88 (3H, m, CH₃(5′)).

### 4-(1-pentene)pyridine

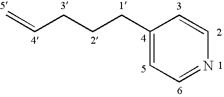

4-Picoline (2.5 g, 26.84 mmol) was dissolved in (12 ml) of dry THF and the solution was cooled to −78° C. with an acetone-dry ice bath. n-Butyllithium (12.35 ml, 30.87 mmol) was added slowly while keeping the internal ° T below −50° C. The reaction was allowed to warm up to room ° T and then stirred at 40 to 45° C. for 2 h. THF (12 ml) was added to dissolve the 4-picolyllithium slurry to give a deep orange solution. The solution was cooled down to 0° C. and carefully added into the solution of 4-bromo-1-butene (3.08 ml, 29.52 mmol) in THF (5.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6× size of column) and the solvent was evaporated by water aspirator. The residue was further purified by flash chromatography (Hex/EtOAc 1.5/1) and the combined fractions were concentrated by water aspirator to give the above compound as a light yellow liquid (3.29 g, 83%). ¹H NMR (200 MHz, d₆-Acetone): δ 8.53 (2H, d, J=6.5 Hz, CH (2,6)), 7.27 (2H, d, J=6.5 Hz, CH(3,5)), 5.92 (1H, m, CH(4′)), 5.07 (2H, m, CH₂(5′)), 2.73 (2H, t, J=7.6 Hz, CH₂(1′)), 2.14 (2H, m, CH₂(2′)), 1.87 (2H, m, CH₂(3′)).

### 4-Isopentylpyridine

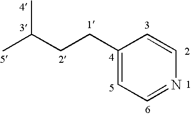

4-Picoline (2.5 g, 26.84 mmol) was dissolved in (12 ml) of dry THF and the solution was cooled to −78° C. with an acetone-dry ice bath, n-Butyllithium (12.35 ml, 30.87 mmol) was added slowly while keeping the internal ° T below −50° C. The reaction was allowed to warm up to room ° T. and then stirred at 40 to 45° C. for 2 h. THF (12 ml) was added to dissolve the 4-picolyllithium slurry to give a deep orange solution. The solution was cooled down to 0° C. and carefully added into the solution of 1-bromo-2-methylpropane (3.21 ml, 29.52 mmol) in THF (5.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6× size of column) and the solvent was evaporated by water aspirator. The residue was further purified by flash chromatography (Hex/EtOAc 1.5/1) and the combined fractions were concentrated by water aspirator to give the above compound as a light yellow liquid (3.92 g, 98%). ¹H NMR (200 MHz, CDCl₃): δ 8.46 (2H, d, J=6.5 Hz, CH (2,6)), 7.11 (2H, J=6.5 Hz, CH(3,5)), 2.60 (2H, t, J=7.6 Hz, CH₂(1′)), 1.52 (1H, m, CH₂(3′)), 1.24 (2H, m, CH₂(2′)), 0.93 (6H, d, CH₃(4′,5′)).

### 4-Hexylpyridine

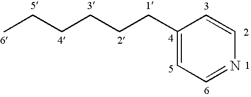

4-Picoline (2.5 g, 26.84 mmol) was dissolved in (12 ml) of dry THF and the solution was cooled to −78° C. with an acetone-dry ice bath. n-Butyllithium (12.35 ml, 30.87 mmol) was added slowly while keeping the internal ° T. below −50° C. The reaction was allowed to warm up to room ° T and then stirred at 40 to 45° C. for 2 h. THF (12 ml) was added to dissolve the 4-picolyllithium slurry to give a deep orange solution. The solution was cooled down to 0° C. and carefully added into the solution of 1-bromopentane (3.66 ml, 29.52 mmol) in THF (5.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6×size of column) and the solvent was evaporated by water aspirator. The residue was further purified by flash chromatography (Hex/EtOAc 1.5/1) and the combined fractions were concentrated by water aspirator to give 5 as a light yellow liquid (2.85 g, 65%). ¹H NMR (200 MHz, CDCl₃): δ 8.46 (2H, d, J=6.5 Hz, CH (2,6)), 7.09 (2H, d, J=6.5 Hz, CH(3,5)), 2.59 (2H, t, J=7.6 Hz, CH₂(1′)), 1.62 (2H, m, CH₂(2′)), 1.24 (6H, m, CH₂(3′,4′,5′)), 0.89 (3H, m, CH₃(6′)).

### 4-Isohexylpyridine

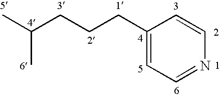

4-Picoline (2.5 g, 26.84 mmol) was dissolved in (12 ml) of dry THF and the solution was cooled to −78° C. with an acetone-dry ice bath. n-Butyllithium (12.35 ml, 30.87 mmol) was added slowly while keeping the internal ° T below −50° C. The reaction was allowed to warm up to room ° T and then stirred at 40 to 45° C. for 2 h. THF (12 ml) was added to dissolve the 4-picolyllithium slurry to give a deep orange solution. The solution was cooled down to 0° C. and carefully added into the solution of 1-bromo-3-methylbutane (3.68 ml, 29.52 mmol) in THF (5.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6×size of column) and the solvent was evaporated by water aspirator. The residue was further purified by flash chromatography (Hex/EtOAc 1.5/1) and the combined fractions were concentrated by water aspirator to give the above compound as a light yellow liquid (3.80 g, 95%). ¹H NMR (200 MHz, CDCl₃): δ 8.46 (2H, d, J=6.5 Hz, CH (2,6)), 7.08 (2H, d, J=6.5 Hz, CH(3,5)), 2.57 (2H, t, J=7.6 Hz, CH₂(1′)), 1.59 (1H, m, CH₂(4′)), 1.24 (4H, m, CH₂(2′,3′)), 0.87 (6H, d, CH₃(5′,6′)).

### 4-Octylpyridine

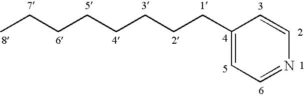

4-Picoline (2.5 g, 26.84 mmol) was dissolved in (12 ml) of dry THF and the solution was cooled to −78° C. with an acetone-dry ice bath. n-Butyllithium (12.35 ml, 30.87 mmol) was added slowly while keeping the internal ° T. below −50° C. The reaction was allowed to warm up to room ° T and then stirred at 40 to 45° C. for 2 h. THF (12 ml) was added to dissolve the 4-picolyllithium slurry to give a deep orange solution. The solution was cooled down to 0° C. and carefully added into the solution of 1-iodoheptane (4.84 ml, 29.52 mmol) in THF (5.0 ml) at −78° C. During the addition, the ° T was kept below −65° C. The reaction was allowed to gradually warm up to room ° T and stirred overnight. The reaction was worked up by adding (1-1.5 ml) of H₂O. The crude was passed through RSF (Rapid Silica Filtration), eluting with EtOAc (volume=6× size of column) and the solvent was evaporated by water aspirator. The residue was further purified by flash chromatography (Hex/EtOAc 1.5/1) and the combined fractions were concentrated by water aspirator to give 7 as a light yellow liquid (3.95 g, 77%). ¹H NMR (200 MHz, CDCl₃): δ 8.47 (2H, d, J=6.5 Hz, CH (2,6)), 7.09 (2H, d, J=6.5 Hz, CH(3,5)), 2.59 (2H, t, J=7.6 Hz, CH₂(1′)), 1.62 (2H, m, CH₂(2′)), 1.27 (10H, m, CH₂(3′,4′,5′,6′,7′)), 0.87 (3H, m, CH₃(8′)).

### 4-Nonylpyridine

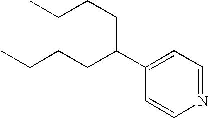

A solution of n-butyllithium (8.80 ml, 22.02 mmol) was added to a solution of diisopropylamine (3.17 ml, 22.55 mmol) in dry THF (112 ml) at −15° C. After stirring for 30 min, 4-picoline (1.00 g, 10.74 mmol) was added dropwise. The resulting red solution was stirred for 1 h at −15° C. and then a solution of 1-bromobutane (2.38 ml, 22.02 mmol) in dry THF (22 ml) was added in one portion. The reaction was allowed to gradually warm up to room ° T and stirred overnight. Et₂O was added and the reaction mixture washed with 1 _{M} NH₄Cl solution (3×50 ml) dried with Na₂SO₄ and evaporated to dryness with the water aspirator. The residue was further purified by flash chromatography (Hex/EtOAc 1.5/1) and the combined fractions were concentrated by water aspirator to give 8 as a light yellow liquid (0.28 g, 13%). ¹H NMR (200 MHz, CDCl₃): δ 8.47 (2H, d, J=6.5 Hz, CH (2,6)), 7.05 (2H, d, J=6.5 Hz, CH(3,5)), 2.46 (1H, m, CH(1′)), 1.57 (4H, m, CH₂(2′)), 1.17 (8H, m, CH₂(3′,4′)), 0.80 (6H, t, CH₃(5′)).

### Synthesis of Bispyridiniuin Compounds

### 1,8-bis(4-pentylpyridinium)octane

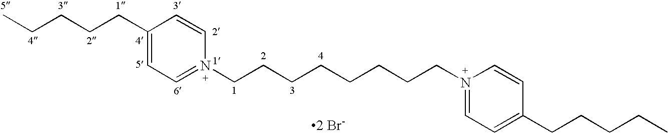

1,8-Dibromooctane (0.50 g, 1.84 mmol) was dissolved in 4-methyl-2-pentanone (2.0 ml) and 4-pentylpyridine (0.68 g, 4.56 mmol) was added. The mixture was stirred at reflux for 18 h under a nitrogen atmosphere, and the solvent was removed under reduced pressure. The crude product was dissolved in methanol (25 ml) and decolourising charcoal was added, then the mixture was boiled for 5 min. The charcoal was removed by filtration and the solvent removed under reduced pressure. The residue was recrystallised from MeOH/Et₂O to yield 10 as a white solid (0.87 g, 83%). ¹H NMR (300 MHz, d₆-DMSO): δ 9.00 (4H, d, J=6.5 Hz, CH (2′,6′)), 8.03 (4H, d, J=6.5 Hz, CH(3′,5′)), 4,54 (4H, t, J=7.6 Hz, CH₂(1)), 2.87 (4H, t, J=7.5 Hz, CH₂(1″)), 1.89 (4H, m, CH₂)), 1.66 (4H, m, CH₂(2″)), 1.29 (16H, m, CH₂(3,4,3″,4″)), 0.86 (6H, m, CH₃(5″)). ¹³C NMR (300 MHz, d₆-DMSO): 163.4, 144.9, 128.5, 60.7, 35.4, 31.5, 31.4, 29.6, 29.0, 26.2, 22.6, 14.6. MS: m/z ESI (positive ion) 205 [M-2Br⁻]²⁺ (100%), 489 [M-⁸¹Br⁻]⁺(15), 491 [M-⁷⁹Br⁻]⁺ (15). Found [M-⁸¹Br⁻]⁺489.2850, [C₂₈H₄₆N₂Br]⁺ requires 489.2839.

### 1,10-bis(4-pentylpyridinium)decane dibromide

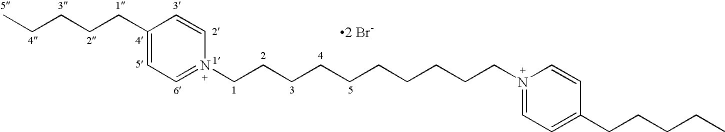

1,10-Dibromodecane (0.50 g, 1.66 mmol) was dissolved in 4-methyl-2-pentanone (2.0 ml) and 4-pentylpyridine (0.69 g, 4.20 mmol) was added. The mixture was stirred at reflux for 18 h under a nitrogen atmosphere, and the solvent was removed under reduced pressure. The crude product was dissolved in methanol (25 ml) and decolourising charcoal was added, then the mixture was boiled for 5 min. The charcoal was removed by filtration and the solvent removed under reduced pressure. The residue was recrystallised from MeOH/Et₂O to yield 11 as a white solid (0.82 g, 82%). ¹H NMR (300 MHz, d₆-DMSO): δ 9.01 (4H, d, J=6.5 Hz, CH(2′,6′)), 8.03 (4H, d, J=6.5 Hz, CH(3′,5′)), 4.55 (4H, t, J=7.5 Hz, CH₂(1)), 2.87 (4H, t, J=7.5 Hz, CH₂(1″)), 1.89 (4H, m, CH₂(2)), 1.65 (4H, m, CH₂(2″)), 1.29 (20H, m, CH₂(3,4,5,3″,4″)), 0.86 (6H, m, CH₃(5″)). ¹³C NMR (300 MHz, d₆-DMSO): 163.4, 144.8, 128.5, 60.7, 35.4, 31.5, 31.4, 29.6, 29.5, 29.2, 26.3, 22.6, 14.6. MS: m/z ESI (positive ion) 219 [M-2Br⁻]²⁺(100%), 437 [M-2Br⁻—H⁺]⁺ (40), 517 [M-⁸¹Br⁻]⁺ (40), 519 [M-⁷⁹Br⁻]⁺ (40). Found [M-⁸¹Br⁻]⁺ 517.3159, [C₃₀H₅₀N₂Br]⁺ requires 517.3152.

### 1,12-bis(3-ethylpyridinium)dodecane dibriomide

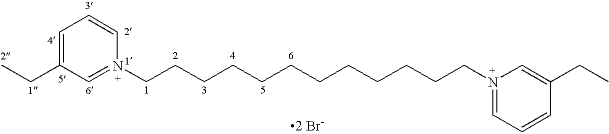

1,12-Dibromododecane (0.50 g, 1.52 mmol) was dissolved in 4-methyl-2-pentanone (2.0 ml) and 3-ethylpyridine (0.41 g, 3.79 mmol) was added. The mixture was stirred at reflux for 18 h under a nitrogen atmosphere, and the solvent was removed under reduced pressure. The crude product was dissolved in methanol (25 ml) and decolourising charcoal was added, then the mixture was boiled for 5 min. The charcoal was removed by filtration and the solvent removed under reduced pressure. The residue was recrystallised from MeOH/Et₂O to yield 13 as a white waxy solid (0.65 g, 79%). ¹H NMR (300 MHz, d₄-MeOD): δ 8.95 (2H, s, CH(6′)), 8.65 (2H, d, J=6.3 Hz, CH(2′)), 8.47 (2H, d, J=6.7 Hz, CH(4′)), 8.02 (2H, m, CH(3′)) 4.62 (4H, t, J=7.4 Hz, CH₂(1)), 2.92 (4H, q, J=7.5 Hz, CH₂(1″)), 2.02 (4H, m, CH₂(2)), 1.33 (22H, m, CH₂(3,4,5,6) & CH₃(2″)). ¹³C NMR (300 MHz, d₄-MeOD): 145.9 (Cq), 145.5 (CH), 144.0 (CH), 142.3 (CH), 128.0 (CH), 61.9 (CH₂), 31.5 (CH₂), 29.5 (CH₂), 29.4 (CH₂), 29.1 (CH₂), 26.2 (CH₂), 25.8 (CH₂), 13.8 (CH₃). MS: m/z ESI (positive ion) 191 [M-2Br⁻]²⁺ (100%), 461 [M-⁸Br⁻]⁺ (25), 463 [M-⁷⁹Br⁻]⁺ (25). Found [M-⁸¹Br⁻]⁺ 461.2521, [C₂₆H₄₂N₂Br]⁺ requires 461.2537.

### 1,12-bis(4-ethylpyridinium)dodecane dibromide

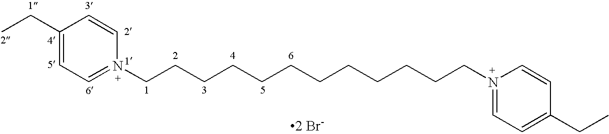

1,12-Dibromododecane (0.50 g, 1.52 mmol) was dissolved in 4-methyl-2-pentanone (2.5 ml) and 4-ethylpyridine (0.41 g, 3.79 mmol) was added. The mixture was stirred at reflux for 24 h under a nitrogen atmosphere, and the solvent was removed under reduced pressure. The crude product was dissolved in methanol (25 ml) and decolourising charcoal was added, then the mixture was boiled for 5 min. The charcoal was removed by filtration and the solvent removed under reduced pressure. The residue was recrystallised from MeOH/Et₂O to yield 14 as a light yellow waxy solid (0.80 g, 98%). ¹H NMR (300 MHz, d₆-DMSO): δ 8.98 (4H, d, J=6.5 Hz, CH(2′,6′)), 8.03 (4H, d, J=6.5 Hz, CH(3′,5′)), 4.53 (4H, t, J=7.4 Hz, CH₂(1)), 2.90 (4H, q, J=7.5 Hz, CH₂(1″)), 1.88 (4H, m, CH₂(2)), 1.26 (22H, m, CH₂(3,4,5,6) & CH₃(2″)). ¹³C NMR (300 MHz, d₆-DMSO): 164.6 (Cq), 144.8 (CH), 128.0 (CH), 60.7 (CH₂), 31.5 (CH₂), 29.8 (CH₂), 29.7 (CH₂), 29.3 (CH₂), 28.8 (CH₂), 26.3 (CH₂), 14.3 (CH₃). MS: m/z ESI (positive ion) 191 [M-2Br⁻]²⁺ (100%), 381 [M-2Br⁻—H⁺]⁺ (40), 461 [M-⁸¹Br⁻]⁺ (20), 463 [M-⁷⁹Br⁻]⁺ (20). Found [M-⁸¹Br⁻]⁺ 461.2526, [C₂₆H₄₂N₂Br]⁺ requires 461.2526.

### 1,12-bis(4-propylpyridinium)dodecane dichloride

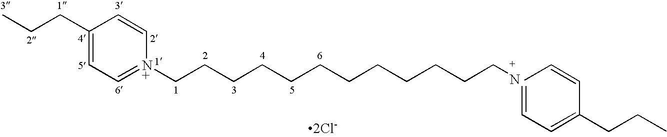

1,12-Dibromododecane (0.50 g, 1.52 mmol) was dissolved in 4-methyl-2-pentanone (2.5 ml) and 4-propylpyridine (0.46 g, 3.79 mmol) was added. The mixture was stirred at reflux for 18 h under a nitrogen atmosphere, and the solvent was removed under reduced pressure. The crude mixture was purified twice by flash chromatography (CHCl₃/MeOH/H₂O 6/4/1). The combined fractions were then passed down a column of Lewatit MP-64 anion resin (Cl⁻), eluting with H₂O. The resulting fractions were combined and the solvent removed under reduced pressure to give the above compound as a light yellow oil (0.4636 g, 64%). ¹H NMR (200 MHz, d₄-MeOD): δ 8.96 (4H, d, J=6.5 Hz, CH(2′,6′)), 8.02 (4H, d, J=6.5 Hz, CH(3′,5′)), 4.53 (4H, t, J=7.6 Hz, CH₂(1)), 2.92 (4H, t, J=7.7 Hz, CH₂(1″)), 1.90 (4H, m, CH₂(2)), 1.83 (4H, m, CH₂(2″)), 1.26 (16H, m, CH₂(3,4,5,6)), 1.05 (6H, t, J=7.4 Hz, CH₃(3″)). ¹³C NMR (300 MHz, d₄-MeOD): 163.9, 144.1, 128.2, 61.2, 37.4, 31.4, 29.5, 29.4, 29.1, 26.2, 23.6, 12.9. MS: m/z ESI (positive ion) 233 [M-2Cl⁻]²⁺ (100%), 465 [M-2Cl⁻—H⁺]⁺ (100). Found [M-2Cl⁻]²⁺ 233.2136, [C₁₆H₂₇N]²⁺ requires 233.2138.

### 1,12-bis(4-isopropylpyridinium)dodecane dibromide

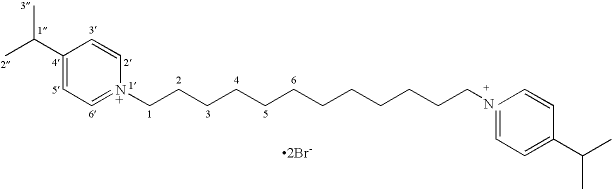

1,12-Dibromododecane (0.50 g, 1.52 mmol) was dissolved in 4-methyl-2-pentanone (2.5 ml) and 4-isopropylpyridine (0.46 g, 3.79 mmol) was added. The mixture was stirred at reflux for 18 h under a nitrogen atmosphere, and the solvent was removed under reduced pressure. The crude product was purified by Al₂O₃ chromatography (neutral, activity II-III), using gradient elution (starting with CHCl₃ to CHCl₃/MeOH 9/1). The resulting fractions were combined and the solvent removed under reduced pressure to give the above compound as a light yellow oil (0.42 g, 50%). ¹H NMR (300 MHz, d₄-MeOD): δ 9.12 (4H, d, J=6.5 Hz, CH(2′,6′)), 8.13 (4H, d, J=6.5 Hz, CH(3′,5′)), 4.75 (4H, t, J=7.6 Hz, CH₂(1)), 3.36 (2H, m, CH(1″)), 2.07 (4H, m, CH₂(2)), 1.37 (28H, m, CH₂(3,4,5,6) & CH₃(2″,3″)). ¹³C NMR (300 MHz, d₄-MeOD): 169.1, 144.7, 126.7, 61.2, 34.6, 31.6, 29.6, 29.5, 29.2, 26.2, 22.1. MS: m/z ESI (positive ion) 205 [M-2Br⁻]²⁺ (100%), 489 [M-⁸¹Br⁻]⁺ (15), 491 [M-⁷⁹Br⁻]⁺ (15). Found [M-⁸¹Br⁻]⁺ 489.2842, [C₂₈H₄₆N₂Br]⁺ requires 489.2839.

### 1,12-bis(4-butylpyridinium)dodecane dichloride

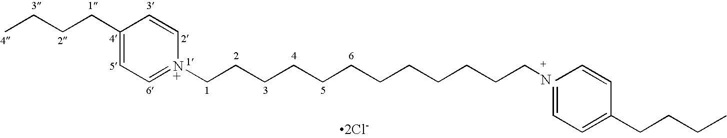

1,12-Dibromododecane (0.25 g, 0.76 mmol) was dissolved in 4-methyl-2-pentanone (2.0 ml) and 4-butylpyridine (0.24 g, 1.74 mmol) was added. The mixture was stirred at reflux for 18 h under a nitrogen atmosphere, and the solvent was removed under reduced pressure. The residue was then diluted with H₂O (15 ml) and washed with dry Et₂O (3×20 ml). The aqueous layer was extracted with CH₂Cl₂ (3×20 ml), then the CH₂Cl₂ layer was concentrated under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.07 g, 37%). ¹H NMR (300 MHz, CDCl₃): δ 9.54 (4H, d, J=6.5 Hz, CH(2′,6′)), 7.78 (4H, d, J=6.5 Hz, CH(3′,5′)), 4.79 (4H, t, J=7.6 Hz, CH₂(1)), 2.75 (4H, t, J=7.5 Hz, CH₂(1″)), 1.92 (4H, m, CH₂(2)), 1.57 (4H, m, CH₂(2″)), 1.26 (12H, m, CH₂(3,4,3″,4″)), 1.10 (8H, m, CH₂(5,6)), 0.96 (6H, m, CH₃(5″)). ¹³C NMR (300 MHz, CDCl₃): 163.0, 145.2, 128.3, 61.2, 35.8, 32.2, 31.9, 29.6, 29.2, 29.0, 26.2, 22.5, 14.0, MS: m/z ESI (positive ion) 219 [M-2Cl⁻]²⁺ (100%), 437 [M-2Cl⁻—H⁺]⁺ (50). Found [M-2Cl⁻]²⁺ 219.1976, [C₁₅H₂₅N]²⁺ requires 219.1981.

### 1,12-bis(2-pentylpyridinium)dodecane dichloride

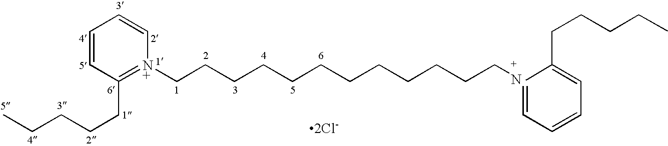

1,12-Dibromododecane (0.20 g, 0.61 mmol) was dissolved in 4-methyl-2-pentanone (2.0 ml) and 2-pentylpyridine (0.20 g, 1.34 mmol) was added. The mixture was stirred at reflux for 36 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.27 g, 82%). ¹H NMR (300 MHz, CDCl₃): δ 9.99 (1H, d, J=6.5 Hz, CH(2′)), 8.42 (1H, m, CH(5′)), 8.01 (1H, m, CH(3′), 7.86 (1H, m, CH(4′), 4.94 (4H, t, J=7.6 Hz, CH₂(1)), 3.10 (4H, t, J=7.5 Hz, CH₂(1″)), 2.88 (4H, m, CH₂(2″)), 1.95 (4H, m, CH₂(2)), 1.80 (4H, m, CH₂(3″)), 1.26 (20H, m, CH₂(3,4,5,6,4″)), 0.92 (6H, m, CH₃(5″)). ¹³C NMR (300 MHz, CDCl₃): 157.8, 148.1, 145.4, 128.8, 126.8, 58.1, 32.9, 32.0, 31.7, 29.1, 29.0, 28.8, 26.4, 22.7, 14.2, 1 signal obscured or overlapping. MS: m/z ESI (positive ion) 233 [M-2Cl⁻]²⁺ (38%), 465 [M-2Cl⁻—H⁺]⁺ (85). Found [M-2Cl⁻]²⁺ 233.2135, [C₁₆H₂₇N]²⁺ requires 233.2138.

### 1,12-bis(3-pentylpyridinium)dodecane dichloride

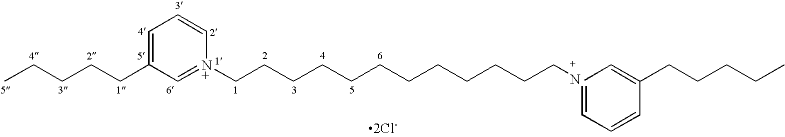

1,12-Dibromododecane (0.20 g, 0.61 mmol) was dissolved in 4-methyl-2-pentanone (2.0 ml) and 3-pentylpyridine (0.20 g, 1.34 mmol) was added. The mixture was stirred at reflux for 20 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.28 g, 85%). ¹H NMR (300 MHz, CDCl₃): δ 9.55 (4H, m, CH(2′,6′)), 8.20 (2H, d, J=6.5 Hz, CH(4′)), 8.07 (2H, m, CH(3′)), 5.00 (4H, m, CH₂(1)), 3.10 (4H, m, CH₂(1″)), 2.89 (4H, m, CH₂(2″)), 2.06 (4H, m, CH₂(2)), 1.72 (4H, m, CH₂(3″)), 1.33 (12H, m, CH₂(3,4,4″)), 1.33 (8H, m, CH₂(5,6)), 0.96 (6H, m, CH₃(5″)). 13C NMR (300 MHz, CDCl₃): 164.2, 144.2, 128.5, 61.2, 35.6, 32.2, 31.4, 29.5, 29.4, 29.1, 26.2, 22.4, 13.3, 1 signal obscured or overlapping. MS: m/z ESI (positive ion) 233 [M-2Cl⁻]²⁺ (18%), 465 [M-2Cl⁻—H⁺]⁺ (10). Found [M-2Cl⁻]²⁺ 233.2136, [C₁₆H₂₇N]²⁺ requires 233.2144.

### 1,12-bis(4-pentylpyridinium)dodecane dichloride

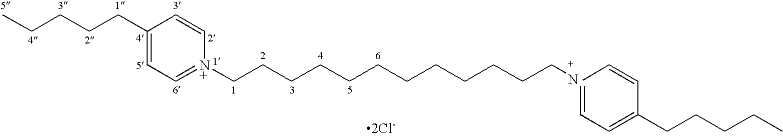

1,12-Dibromododecane (0.50 g, 1.52 mmol) was dissolved in 4-methyl-2-pentanone (2.0 ml) and 4-pentylpyridine (0.56 g, 3.79 mmol) was added. The mixture was stirred at reflux for 18 h under a nitrogen atmosphere, and the solvent was removed under reduced pressure. The residue was then diluted with H₂O (15 ml) and washed with dry Et₂O (3×20 ml). The aqueous layer was extracted with CH₂Cl₂ (3×20 ml), then the CH₂Cl₂ layer was concentrated under reduced pressure. The residue was passed down a column of Lewatit MP-64 anion resin (Cl⁻), eluting with EtOH. The resulting fractions were combined and the solvent removed under reduced pressure to give the above compound as a light brown waxy oil (0.68 g, 83%). ¹H NMR (300 MHz, d₄-MeOD): δ 8.94 (4H, d, J=6.5 Hz, CH(2′,6′)), 8.02 (4H, d, J=6.5 Hz, CH(3′,5′)), 4.65 (4H, t, J=7.6 Hz, CH₂(1)), 3.00 (4H, t, J=7.5 Hz, CH₂(1″)), 2.03 (4H, m, CH₂(2)), 1.79 (4H, m, CH₂(2″)), 1.26 (24H, m, CH₂(3,4,5,6,3″,4″)), 0.96 (6H, m, CH₃(5″)). ¹³C NMR (300 MHz, d₄-MeOD): 164.2, 144.2, 128.5, 61.2, 35.6, 32.2, 31.4, 29.5, 29.4, 29.1, 26.2, 22.4, 13.3, 1 signal obscured or overlapping. MS: m/z ESI (positive ion) 233 [M-2Cl⁻]²⁺ (100%), 465 [M-2Cl⁻—H⁺]⁺ (100). Found [M-2Cl⁻]²⁺ 233.2136, [C₁₆H₂₇N]²⁺ requires 233.2138.

### 1,12-bis[4-(1-pentene)pyiridinium]dodecane dichloride

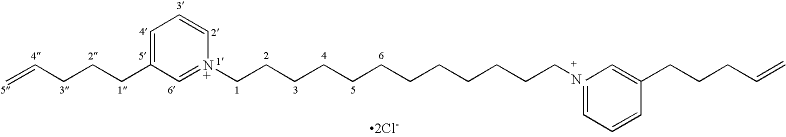

1,12-Dibromododecane (0.20 g, 0.61 mmol) was dissolved in 4-methyl-2-pentanone (2.0 ml) and 4-(1-pentene)pyridine (0.21 g, 1.40 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.24 g, 74%). ¹H NMR (200 MHz, CDCl₃): δ 9.58 (4H, d, J=6.5 Hz, CH(2′,6′)), 7.86 (4H, d, J=6.5 Hz, CH(3′,5′)), 5.69 (1H, m, CH(4″)), 5.07 (2H, m, CH₂(5″)), 4.94 (4H, m, CH₂(1)), 2.87 (4H, t, J=7.5 Hz, CH₂(1″)), 2.14 (8H, m, CH₂(2, 3″)), 1.83 (4H, m, CH₂(2″)), 1.27 (16H, m, CH₂(3,4,5,6)). ¹³C NMR (300 MHz, d₄-MeOD): 162.5, 144.6, 137.2, 128.1, 115.8, 60.4, 34.9, 32.8, 31.8, 29.1, 28.8, 28.6, 25.8, 14.1. MS: m/z ESI (positive ion) 231 [M-2Cl⁻]²⁺ (100%), 462 [M-2Cl⁻—H⁺]⁺ (100). Found [M-2Cl⁻]²⁺ 231.1978, [C₁₆H₂₅N]²⁺ requires 231.1981.

### 1,12-bis(4-isopentylpyridinium)dodecane dichloride

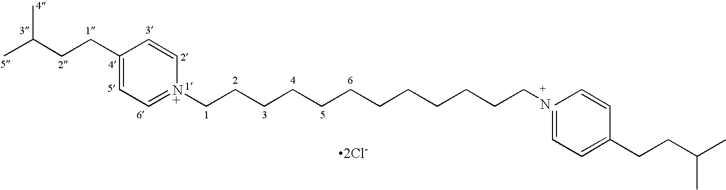

1,12-Dibromododecane (0.20 g, 0.61 mmol) was dissolved in 4-methyl-2-pentanone (2.0 ml) and 4-isopentylpyridine (0.21 g, 1.40 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.32 g, 98%). ⁻H NMR (300 MHz, d₄-MeOD): δ 9.10 (4H, d, J=6.5 Hz, CH(2′,6′)), 8.08 (4H, d, J=6.5 Hz, CH(3′,5′)), 4.74 (4H, t, J=7.6 Hz, CH₂(1)), 3.02 (4H, t, J=7.5 Hz, CH₂(1″)), 2.04 (4H, m, CH₂(2)), 1.67 (4H, m, CH₂(2″)), 1.38 (1H, m, CH(3″)), 1.29 (16H, m, CH₂(3,4,5,6)), 0.96 (12H, m, CH₃(4″,5″)). ¹³C NMR (300 MHz, d₄-MeOD): 164.2, 144.4, 128.3, 61.2, 39.0, 33.7, 31.6, 29.6, 29.5, 29.2, 28.1, 26.2, 22.1, 1 signal obscured or overlapping. MS: m/z ESI (positive ion) 233 [M-2Cl⁻]²⁺ (100%), 465 [M-2Cl⁻—H⁺]⁺ (33). Found [M-2Cl⁻]²⁺ 233.2136, [C₁₆H₂₇N]²⁺ requires 233.2138.

### 1,12-bis(4-hexylpyridinium)dodecane dichloride

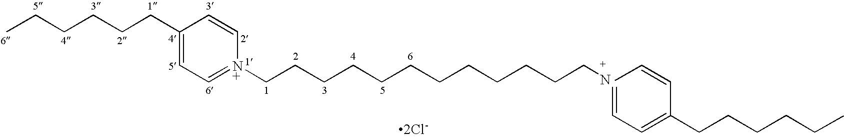

1,12-Dibromododecane (0.75 g, 2.28 mmol) was dissolved in 4-methyl-2-pentanone (2.5 ml) and 4-hexylpyridine (0.93 g, 5.70 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.95 g, 74%). ¹H NMR (300 MHz, CDCl₃): δ 9.59 (4H, d, J=6.5 Hz, CH(2′,6′)), 7.84 (4H, d, J=6.5 Hz, CH(3′,5′)), 4.91 (4H, t, J=7.6 Hz, CH₂(1)), 2.84 (4H, t, J=7.5 Hz, CH₂(1″)), 2.05 (4H, m, CH₂(2)), 1.66 (4H, m, CH₂(2″)), 1.25 (28H, m, CH₂(3,4,5,6,3″,4″,5″)), 0.86 (6H, m, CH₃(6″)). ¹³C NMR (300 MHz, CDCl₃): 163.2, 145.1, 128.3, 61.3, 36.3, 32.3, 31.7, 29.9, 29.2, 29.1, 28.8, 26.1, 22.8, 14.4, 1 signal obscured or overlapping. MS: m/z ESI (positive ion) 247 [M-2Cl⁻]²⁺ (100%), 493 [M-2Cl⁻—H⁺]⁺ (35). Found [M-2Cl⁻]²⁺ 247.2294, [C₁₇H₂₉N]²⁺ requires 247.2290.

### 1,12-bis(4-isohexylpyridinium)dodecane dichloride)

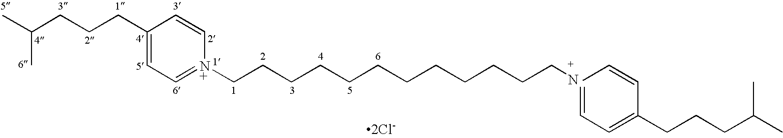

1,12-Dibromododecane (0.20 g, 0.61 mmol) was dissolved in 4-methyl-2-pentanone (2.0 ml) and 4-isohexylpyridine (0.21 g, 1.40 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was passed down a column of Lewatit MP-64 anion resin (Cl⁻), eluting with EtOH. The resulting fractions were combined and the solvent removed under reduced pressure to give the above compound as a light yellow waxy oil (0.34 g, 97%). ¹H NMR (300 MHz, d₄-MeOD): δ 8.98 (4H, d, J=6.5 Hz, CH(2′,6′)), 8.02 (4H, d, J=6.5 Hz, CH(3′,5′)), 4.66 (4H, t, J=7.6 Hz, CH₂(1)), 2.96 (4H, t, J=7.5 Hz, CH₂(1″)), 2.03 (4H, m, CH₂(2)), 1.78 (4H, m, CH₂(2″)), 1.63 (1H, m, CH(4″)), 1.35 (8H, m, CH₂(3″,3)), 1.29 (12H, m, CH₂(4,5,6)), 0.90 (12H, m, CH₃(5″,6″)). ¹³C NMR (300 MHz, d₄-MeOD): 164.0, 144.3, 128.7, 61.2, 38.5, 35.8, 31.5, 29.6, 29.5, 29.2, 28.0, 27.8, 26.2, 22.1. MS: m/z ESI (positive ion) 247 [M-2Cl⁻]²⁺ (100%), 493 [M-2Cl⁻—H⁺]⁺(35). Found [M-2Cl⁻]²⁺ 247.2302, [C₁₇H₂₉N]²⁺ requires 247.2295.

### 1,12-bis(4-octylpyridinium)dodecane dichloride

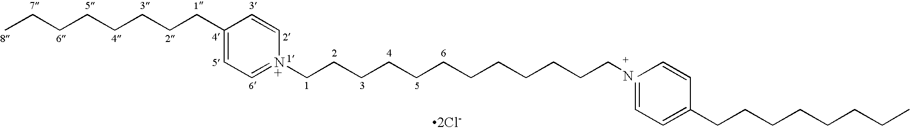

1,12-Dibromododecane (0.20 g, 0.61 mmol) was dissolved in 4-methyl-2-pentanone (2.0 ml) and 4-octylpyridine (0.27 g, 1.40 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.36 g, 95%). ¹H NMR (300 MHz, CDCl₃): δ 9.36 (4H, d, J=6.5 Hz, CH(2′,6′)), 7.58 (4H, d, J=6.5 Hz, CH(3′,5′)), 4.58 (4H, t, J=7.6 Hz, CH₂(1)), 2.53 (4H, t, J=7.5 Hz, CH₂(1″)), 1.73 (4H, m, CH₂(2)), 1.35 (4H, m, CH₂(2″)), 0.90 (24H, m, CH₂(3,4,5,6,3″4″,5″,6″,7″)), 0.52 (6H, m, CH₃(8″)). ¹³C NMR (300 MHz, CDCl₃): 168.5, 150.7, 133.8, 66.7, 41.7, 37.7, 37.5, 35.4, 35.0, 34.8, 34.6, 31.7, 28.4, 19.8, 3 signals obscured or overlapping. MS: m/z ESI (positive ion) 275 [M-2Cl⁻]²⁺ (100%), 549 [M-2Cl⁻—H⁺]⁺ (35).Found [M-2Cl⁻]²⁺ 275.2609, [Cl₉H₃₃N]²⁺ requires 275.2608.

### 1,12-bis(4-nonylpyridinium)dodecane dichloride

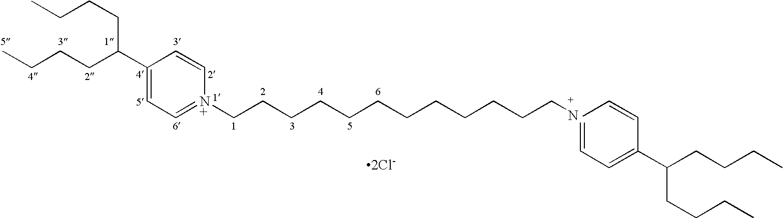

1,12-Dibromododecane (0.16 g, 0.48 mmol) was dissolved in 4-methyl-2-pentanone (2.0 ml) and 4-nonylpyridine (0.23 g, 1.10 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The residue was passed down a column of Lewatit MP-64 anion resin (Cl⁻), eluting with EtOH. The resulting fractions were combined and the solvent removed under reduced pressure to give the above compound as a light yellow waxy oil (0.15 g, 38%). ¹H NMR (200 MHz, CDCl₃): δ 9.74 (4H, d, J=6.5 Hz, CH(2′,6′)), 7.75 (4H, d, J=6.5 Hz, CH(3′,5′)), 4.89 (4H, t, J=7.6 Hz, CH₂(1)), 2.72 (2H, m, CH(1″)), 2.02 (4H, m, CH₂(2)), 1.65 (8H, m, CH₂(2″)), 1.26 (32H, m, CH₂(3,4,5,6,3″,4″)), 0.77 (12H, m, CH₃(5″)). ¹³C NMR (300 MHz, CDCl₃): 167.0, 145.4, 127.6, 61.2, 46.8, 35.8, 32.2, 29.8, 29.2, 29.1, 28.8, 26.2, 22.9, 14.1. MS: m/z ESI (positive ion) 289 [M-2Cl⁻]²⁺ (100%), 577 [M-2Cl⁻—H⁺]⁺(50).Found [M-2Cl⁻]²⁺ 289.2759, [C₂₀H₃₅N]²⁺ requires 289.2764.

### 1,12-bis(4-benzylpyridium)dodecane dichloride

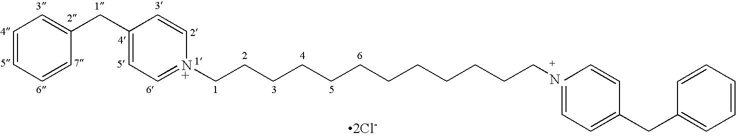

1,12-Dibromohexadecane (0.50 g, 1.52 mmol) was dissolved in 4-methyl-2-pentanone (2.5 ml) and 4-benzylpyridine (0.64 g, 3.80 mmol) was added. The mixture was stirred at reflux for 20 h under a nitrogen atmosphere, and the solvent was removed under reduced pressure. The residue was then diluted with H₂O (˜15 ml) and washed with dry Et₂O (3×20 ml). The aqueous layer was extracted with CH₂Cl₂ (4×30 ml), then the CH₂Cl₂ layer was concentrated under reduced pressure. The residue was purified by 3×Al₂O₃ chromatography (neutral, activity II-III), using gradient elution (starting with CHCl3/MeOH=2% to 10%). The combined fractions were then passed down a column of Lewatit MP-64 anion resin (Cl⁻), eluting with EtOH. The resulting fiactions were combined and the solvent removed under reduced pressure to give the above compound as a brown waxy oil (0.32 g, 36%). ¹H NMR (200 MHz, d₄-MeOD): δ 8.98 (4H, d, J=6.5 Hz, CH(2′,6′)), 8.04 (4H, d, J=6.5 Hz, CH(3′,5′)), 7.49 (10H, m, CH(3″,4″,5″,6″,7″)), 4.68 (4H, t, J=7.5 Hz, CH₂(1)), 4.45 (4H, s, CH₂(1″)), 2.03 (4H, m, CH₂(2)), 2.11 (4H, m, CH₂(2″)), 1.42 (16H, m, CH₂(3,4,5,6)). ¹³C NMR (300 MHz, CDCl₃): 162.0, 145.3, 129.8, 129.7, 128.5, 128.2, 61.4, 42.0, 32.2, 30.1, 29.0, 28.9, 28.7, 26.1. MS: m/z ESI (positive ion) 253 [M-2Cl⁻]²⁺ (100%), 506 [M-2Cl⁻—H⁺]⁺ (65) [M-2Cl⁻]²⁺ 253.1824, [C₁₈H₂₃N]²⁺ requires 253.1825.

### 1,12-bis(4-benzoylpyridinium)dodecane dichloride

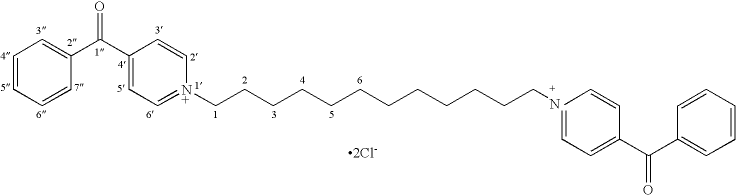

1,12-Dibromohexadecane (0.50 g, 1.52 mmol) was dissolved in 4-methyl-2-pentanone (2.0 ml) and 4-benzoylpyridine (0.70 g, 3.80 mmol) was added. The mixture was stirred at reflux for 20 h under a nitrogen atmosphere, and the solvent was removed under reduced pressure. The residue was then diluted with H₂O (˜15 ml) and washed with dry Et₂O (3×20 ml). The aqueous layer was extracted with CH₂Cl₂ (4×30 ml), then the CH₂Cl₂ layer was concentrated under reduced pressure. The residue was purified by 4×Al₂O₃ chromatography (neutral, activity II-III), using gradient elution (starting with CHCl₃/MeOH=2% to 10%). The combined fractions were then passed down a column of Lewatit MP-64 anion resin (Cl⁻), eluting with EtOH. The resulting fractions were combined and the solvent removed under reduced pressure to give the above compound as a brown-reddish waxy oil (0.72 g, 78%). ¹H NMR (200 MHz, d₄-MeOD): δ 8.98 (4H, d, J=6.5 Hz, CH(2′,6′)), 8.04 (4H, d, J=6.5 Hz, CH(3′,5′)), 7.49 (10H, m, CH(3″,4″,5″,6″,7″)), 4.68 (4H, t, J=7.5 Hz, CH₂(1)), 4.45 (4H, s, CH₂(1″)), 2.03 (4H, m, CH₂(2)), 2.11 (4H, m, CH₂(2″)), 1.42 (16H, m, CH₂(3,4,5,6)), ¹³C NMR (300 MHz, CDCl₃): 162.0, 145.3, 129.8, 129.7, 128.5, 128.2, 61.4, 42.0, 32.2, 30.1, 29,0, 28.9, 28.7, 26.1. MS: in/z ESI positive ion) 261 [M-2Cl⁻]²⁺ (100%), 521 [M-2Cl⁻—H⁺]⁺ (90) Found [M-2Cl⁻]²⁺ 261.2454, [C₁₈H₃₁N]²⁺ requires 261.2451.

### 1,12-bis(4-propylphenylpyridinium)dodecane dichloride

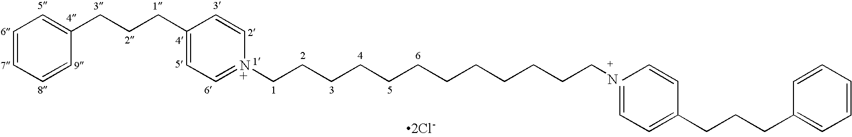

1,12-Dibromohexadecane (0.20 g, 0.61 mmol) was dissolved in 4-methyl-2-pentanone (2.5 ml) and 4-propylphenylpyridine (0.26 g, 1.34 mmol) was added. The mixture was stirred at reflux for 18 h under a nitrogen atmosphere, and the solvent was removed under reduced pressure. The crude was triturated with Et₂O (8×10 ml), and the solvent was removed under reduced pressure. The residue was purified by Al₂O₃ chromatography (neutral, activity II-III), using gradient elution (starting with CHCl₃/MeOH=2% to 10%). The combined fractions were then passed down a column of Lewatit MP-64 anion resin (Cl⁻), eluting with EtOH. The resulting fractions were combined and the solvent removed under reduced pressure to give the above compound as a yellow waxy oil (0.34 g, 88%). ¹H NMR (300 MHz, CDCl₃): δ 9.57 (4H, d, J=6.5 Hz, CH(2′,6′)), 7.82 (4H, d, J=6.5 Hz, CH(3′,5′)), 7.21 (10H, m, CH(5″,6″,7″,8″,9″)), 4.87 (4H, t, J=7.5 Hz, CH₂(1)), 2.83 (4H, m, CH₂(1″)), 2.66 (4H, m, CH₂(3″)), 2.05 (8H, m, CH₂(2,2″)), 1.42 (16H, m, CH₂(3,4,5,6)). ¹³C NMR (300 MHz, CDCl₃): 162.6, 145.2, 140.8, 129.0, 128.8, 128.4, 126.7, 61.3, 35.5, 32.2, 31.3, 29.2, 28.9, 26.2, 2 signals obscured or overlapping. MS: m/z ESI (positive ion) 281 [M-2Cl⁻]²⁺ (100%), 561 [M-2Cl⁻—H⁺]⁺(100). Found [M-2Cl⁻]²⁺ 281.2144, [C₂₀H₂₇N]²⁺ requires 2281.2138.

### 1,12-bis(4-pyridiizium propanol)dodecane dichloride

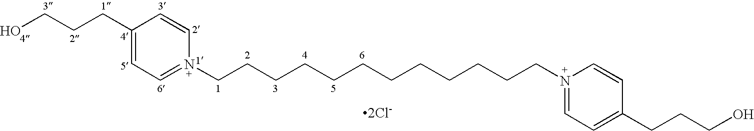

1,12-Dibromohexadecane (0.50 g, 1.52 mmol) was dissolved in CH₃CN (2.5 ml) and 4-pyridine propanol (0.52 g, 3.80 mmol) was added. The mixture was stirred at reflux for 18 h under a nitrogen atmosphere, and the solvent was removed under reduced pressure. The residue was purified by Al₂O₃ chromatography (neutral, activity II-III), using gradient elution (starting with CHCl₃/MeOH=8% to 20%). The combined fractions were then passed down a column of Lewatit MP-64 anion resin (Cl⁻), eluting with EtOH. The resulting fractions were combined and the solvent removed under reduced pressure to give the above compound as a brown waxy oil (0.72 g, 92%). ¹H NMR (200 MHz, d₄-MeOD): δ 8.90 (4H, d, J=6.5 Hz, CH(2′,6′)), 8.00 (4H, d, J=6.5 Hz, CH(3′,5′)), 4.62 (4H, t, J=7.5 Hz, CH₂(1)), 3.63 (4H, t, J=7.5 Hz, CH₂(3″)), 3.05 (4H, t, J=7.5 Hz, CH₂(1″)), 1.97 (8H, m, CH₂(2,2″)), 1.25 (16H, m, CH₂(3,4,5,6)). ¹³C NMR (300 MHz, d₄-MeOD): 163.9, 144.2, 128.4, 61.3, 60.7, 32.5, 32.3, 31.4, 29.5, 29.4, 29.1, 26.2. MS: m/z ESI (positive ion) 221 [M-2Cl⁻]²⁺ (100%), 442 [M-2Cl⁻—H⁺]⁺ (13). Found [M-2Cl⁻]²⁺ 221.1770, [C₁₄H₂₃NO]²⁺ requires 221.1774.

### 1,14-bis(4-pentylpyridinium)tetradecane dichloride

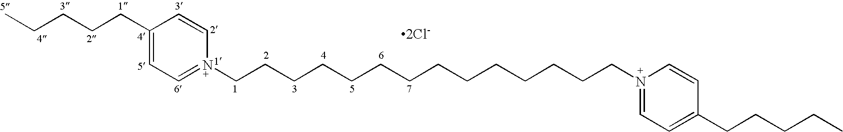

1,4-Dibromotetradecane (0.50 g, 1.40 mmol) was dissolved in 4-methyl-2-pentanone (2.0 ml) and 4-pentylpyridine (0.52 g, 3.50 mmol) was added. The mixture was stirred at reflux for 18 h under a nitrogen atmosphere, and the solvent was removed under reduced pressure. The residue was then diluted with H₂O (˜15 ml) and washed with dry Et₂O (3×20 ml). The aqueous layer was extracted with CH₂Cl₂ (3×20 ml), then the CH₂Cl₂ layer was concentrated under reduced pressure. The residue was purified by Al₂O₃ chromatography (neutral, activity II-III); using gradient elution (starting with CHCl₃/MeOH=2% to 10%). The combined fractions were then passed down a column of Lewatit MP-64 anion resin (ClI), eluting with EtOH. The resulting fractions were combined and the solvent removed under reduced pressure to give the above compound as a light brown waxy oil (0.67 g, 85%). ¹H NMR (300 MHz, d₄-MeOD): δ 8.94 (4H, d, J=6.5 Hz, CH(2′,6′)), 8.02 (4H, d, J=6.5 Hz, CH(3′,5′)), 4.65 (4H, t, J=7.5 Hz, CH₂(1)), 3.02 (4H, t, J=7.5 Hz, CH₂(1″)), 2.07 (4H, m, CH₂(2)), 1.82 (4H, m, CH₂(2″)), 1.42 (28H, m, CH₂(3,4,5,6,7,3″,4″)), 0.98 (6H, m, CH₃(5″)). ¹³C NMR (300 MHz, d₄-MeOD): 164.2, 144.1, 128.5, 61.2, 35.6, 31.6, 31.5, 29.8, 29.7, 29.6, 29.2, 26.2, 22.5, 13.6, 1 signal -obscured or overlapping. MS: m/z ESI (positive ion) 233 [M-2Cl⁻]²⁺ (100%), 465 [M-2Cl⁻—H⁺]⁺ (100) Found [M-2Cl⁻]²⁺ 233.2136, [Cl₆H₂₇N]²⁺ requires 233.2138.

### 1,16-bis(4-pentylpyridinium)hexadecane dichloride

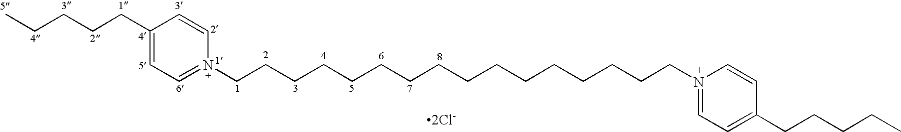

1,16-Dibromohexadecane (0.20 g, 0.49 mmol) was dissolved in 4-methyl-2-pentanone (2.0 ml) and 4-pentylpyridine (0.18 g, 1.24 mmol) was added. The mixture was stirred at reflux for 18 h under a nitrogen atmosphere, and the solvent was removed under reduced pressure. The residue was then diluted with H₂O (˜15 ml) and washed with dry Et₂O (3×20 ml). The aqueous layer was extracted with CH₂Cl₂ (3×20 ml), then the CH₂Cl₂ layer was concentrated under reduced pressure. The residue was purified by Al₂O₃ chromatography (neutral, activity II-III), using gradient elution (starting with CHCl₃/MeOH=2% to 10%). The combined fractions were then passed down a column of Lewatit MP-64 anion resin (Cl⁻), eluting with EtOH. The resulting fractions were combined and the solvent removed under reduced pressure to give the above compound is as a light brown waxy oil (0.25 g, 86%). ¹H NMR (300 MHz, d₄-MeOD): δ 8.99 (4H, d, J=6.5 Hz, CH(2′,6′)), 8.02 (4H, d, J=6.5 Hz, CH(3′,5′)), 4.66 (4H, t, J=7.5 Hz, CH₂(1)), 2.98 (4H, t, J=7.5 Hz, CH₂(1″)), 2.03 (4H, m, CH₂(2)), 1.79 (4H, m, CH₂(2″)), 1.41 (32H, m, CH₂(3,4,5,6,7,8,3″,4″)), 0.93 (6H, m, CH₃(5″)). ¹³C NMR (300 MHz, d₄-MeOD): 164.0, 144.3, 128.5, 61.2, 35.6, 31.6, 31.5, 29.9, 29.8, 29.6, 29.5, 29.2, 26.3, 20 22.5, 13.5, 1 signal obscured or overlapping. MS: in/z ESI positive ion) 261 [M-2Cl⁻]²⁺ (100%), 521 [M-2Cl⁻—H⁺]⁺ (90). Found [M-2Cl⁻]²⁺ 261.2454, [C₁₈H₃₁N]²⁺ requires 261.2451.

TABLE 1
In vitro antifungal activity of bis-(alkylpyridinium)alkane salts
R—(CH₂)ₙ—R •2Cl⁻

| | | | MIC (μM) | |
|---|---|---|---|---|
| | | | C. neoformans | C. albicans |
| Entry | R | n | ATCC 90112 | ATCC 10231 |
| 1 | 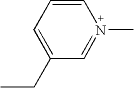 | 12 | 88 | 44 |
| 2 | 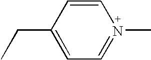 | 12 | 44 | 11 |
| 3 | 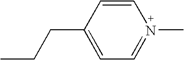 | 12 | 44 | 11 |
| 4 | 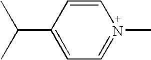 | 12 | 44 | 22 |
| 5 | 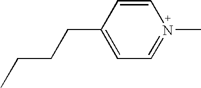 | 12 | 11 | 5.5 |
| 6 | 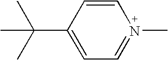 | 12 | 11 | 22 |
| 7 | 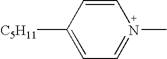 | 8 | 44 | 2.7 |
| 8 | 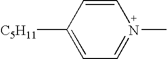 | 10 | 11 | 2.7 |
| 9 | 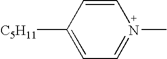 | 12 | 2.7 | 2.7 |
| 10 | 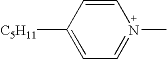 | 14 | 1.4 | 0.7 |
| 11 | 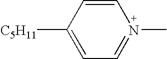 | 16 | 0.7 | 0.7 |
| 12 | 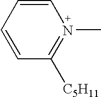 | 12 | 11 | 2.7 |
| 13 | 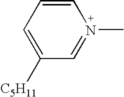 | 12 | 11 | 5.5 |
| 14 | 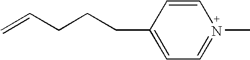 | 12 | 22 | 5.5 |
| 15 | 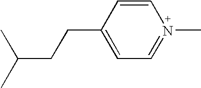 | 12 | 5.5 | 2.7 |
| 16 | 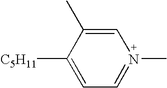 | 12 | n.d | 2.7 |
| 17 | 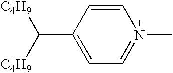 | 12 | 1.4 | 1.4 |
| 18 | 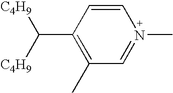 | 12 | n.d. | 1.4 |
| 19 | 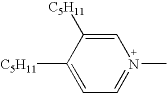 | 12 | n.d. | 1.4 |
| 20 | 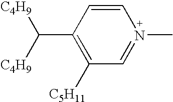 | 12 | n.d | 5.5 |
| 21 | 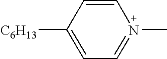 | 12 | 1.4 | 1.4 |
| 22 | 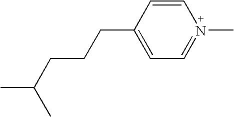 | 12 | 2.7 | 2.7 |
| 23 | 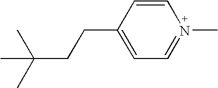 | 12 | n.d | 5.5 |
| 24 | 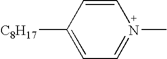 | 12 | 2.7 | 2.7 |
| 25 | 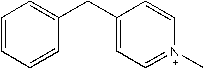 | 12 | 22 | 11 |
| 26 | 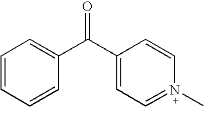 | 12 | 22 | 11 |
| 27 | 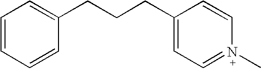 | 12 | 2.7 | 2.7 |
| 28 | 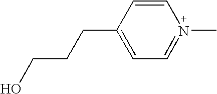 | 12 | 88 | 88 |
| 29 | 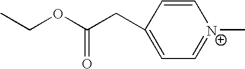 | 12 | nd | 44 |
| 30 | 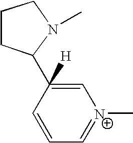 | 12 | >50 | >50 |

TABLE 1a
Antifungal activity of bis(alkylpyridinium)ethylene glycol derivatives

| | MIC (μM) | |
|---|---|---|
| | C. neoformans | C. albicans |
| | ATCC 90112 | ATCC 10231 |
| 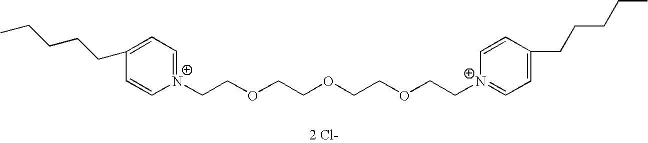 | 44-88 | >175 |
| 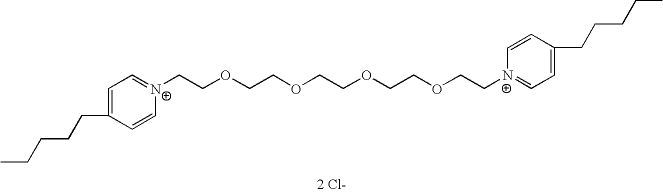 | 88 | >175 |

TABLE 1b
Broad antifungal activity spectrum of some bis(alkylpyridinium)alkane salts

| | | | | | | | | | | |
|---|---|---|---|---|---|---|---|---|---|---|
| compounds→ | i | ii | iii | iv | v | vi | vii | viii | ix | x |
| (see below) | MIC | MIC | MIC | MIC | MIC | MIC | MIC | MIC | MIC | MIC |
| ID | (μM) | (μM) | (μM) | (μM) | (μM) | (μM) | (μM) | (μM) | (μM) | (μM) |

YEASTS

| | | | | | | | | | | | |
|---|---|---|---|---|---|---|---|---|---|---|---|
| C. albicans | ATCC 90028 | 2.75 | 2.75 | 5.5 | 2.75 | 1.4 | 5.5 | 2.75 | 2.75 | 2.75 | 2.75 |
| C. albicans | ATCC 10231 | 2.75 | 2.75 | 5.5 | 2.75 | 1.4 | 5.5 | 2.75 | 2.75 | 2.75 | 2.75 |
| C. krusei | ATCC 6258 | 11 | 44 | 44 | 11 | 5.5 | 44 | 22 | 11 | 2.75 | 5.5 |
| C. krusei | ATCC 9258 | 11 | 44 | 22 | 22 | nd | nd | nd | nd | 2.75 | n.d. |
| C. krusei | 04-202328 | 22 | 44 | 44 | 22 | 5.5 | 44 | 22 | 11 | 2.75 | 5.5 |
| C. parapsilosis | ATCC 22019 | 1.4 | 1.4 | 2.75 | 2.75 | 1.4 | 2.75 | 1.4 | 1.4 | 2.75 | 2.75 |
| C. parapsilosis | 04-202398 | 87.5 | 87.5 | >87.5 | 87.5 | 44 | 87.5 | 87.5 | 44 | 11 | 22 |
| C. glabrata | 04-202345 | 2.75 | 0.7 | 2.75 | 2.75 | 1.4 | 1.4 | 1.4 | 1.4 | 1.4 | 2.75 |
| C. tropicalis | 04-202361 | 1.4 | 1.4 | 5.5 | 2.75 | 1.4 | 2.75 | 1.4 | 1.4 | 2.75 | 2.75 |
| C. neoformans | 05-202523 | 87.5 | 87.5 | 22 | 22 | 11 | 44 | 44 | 22 | 11 | 5.5 |
| C. neoformans | ATCC 90112 | 2.75 | 11 | 5.5 | 22 | 1.4 | 2.75 | 2.75 | 1.4 | 2.75 | 2.75 |
| C. gattii | 05-200122 | 1.4 | 2.75 | 2.75 | 2.75 | 1.4 | 2.75 | 2.75 | 1.4 | 2.75 | 2.75 |
| MOULDS | | | | | | | | | | | |
| A. fumigatus | ATCC | 87.5 | >87.5 | >87.5 | >87.5 | 22 | >87.5 | 44 | 44 | 11 | 22 |
| | 204305 | | | | | | | | | | |
| A. fumigatus | 05-203114 | 87.5 | >87.5 | >87.5 | >87.5 | 22 | >87.5 | 22 | 44 | 11 | 22 |
| A. flavus | 05-203758 | 87.5 | >87.5 | >87.5 | >87.5 | 44 | >87.5 | >87.5 | 44 | 22 | 87.5 |
| A. flavus | ATCC | 44 | >87.5 | >87.5 | >87.5 | 22 | >87.5 | 87.5 | 22 | 11 | 22 |
| | 204304 | | | | | | | | | | |
| A. terreus | 05-202844 | 11 | 1.4 | 5.5 | 2.75 | 1.4 | 87.5 | 2.75 | 1.4 | 1.4 | 5.5 |
| S. prolificans | 05-203060 | 2.75 | 2.75 | 5.5 | 2.75 | 44 | 2.75 | >87.5 | 2.75 | 2.75 | 5.5 |
| S. apiospermum | 05-203100 | 5.5 | 2.75 | 22 | 22 | 87.5 | 11 | >87.5 | 11 | 5.5 | 2.75 |
| Cunninghamella berth | 99-12187 | 87.5 | 87.5 | >87.5 | >87.5 | 22 | >87.5 | 44 | n.d. | 11 | 22 |
| Absidia corymbifera | 04-200170 | >87.5 | >87.5 | >87.5 | >87.5 | 87.5 | >87.5 | >87.5 | 87.5 | 22 | 44 |
| Rhizopus oryzae | 05-203111 | 2.75 | 5.5 | 11 | 11 | 1.4 | 11 | 1.4 | 2.75 | 2.75 | 5.5 |
| Exophiala spinifera | 03-200803 | 0.7 | 0.7 | 2.75 | 1.4 | 2.75 | 1.4 | 2.75 | 1.4 | 2.75 | 2.75 |
| Exophiala jeanselmei | 03-200268 | 1.4 | 1.4 | 5.5 | 11 | 1.4 | 2.75 | 2.7C5 | 2.75 | 2.75 | 5.5 |
| Paecilomyces lilacinus | 05-200601 | >87.5 | >87.5 | >87.5 | >87.5 | 87.5 | >87.5 | >87.5 | >87.5 | 22 | 87.5 |
| Fusarium solani | 04-203102 | 87.5 | 44 | 87.5 | 87.5 | 22 | >87.5 | 44 | 22 | 22 | 44 |

Compounds for Table 1b:

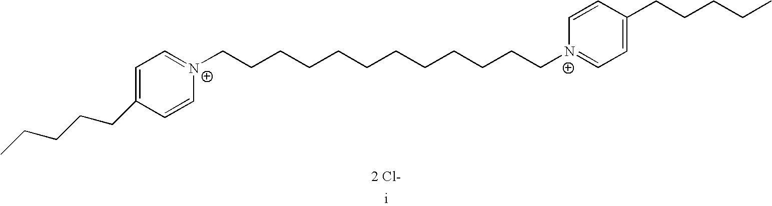

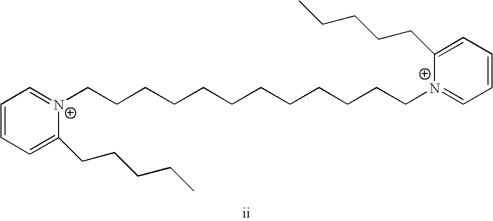

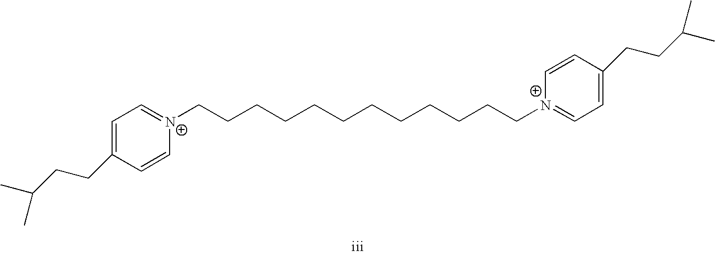

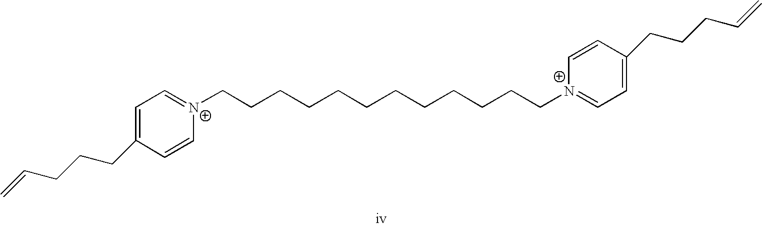

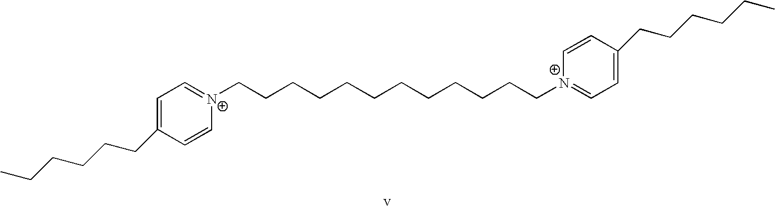

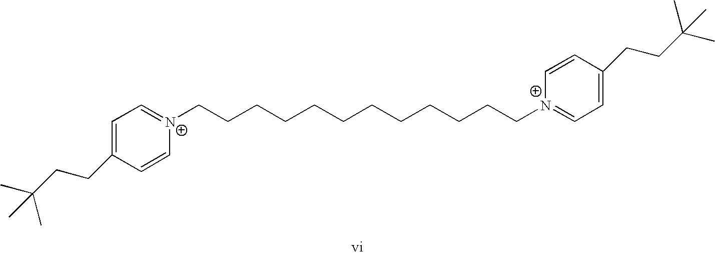

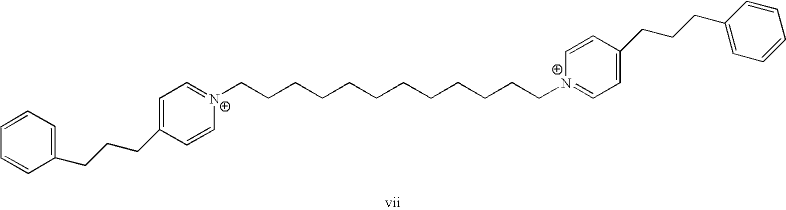

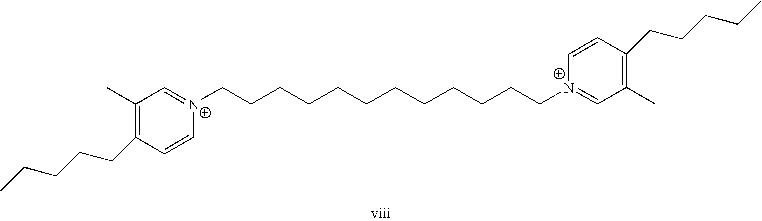

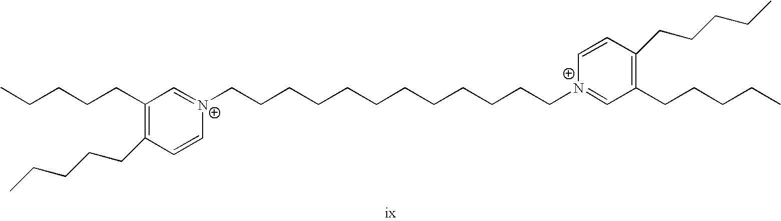

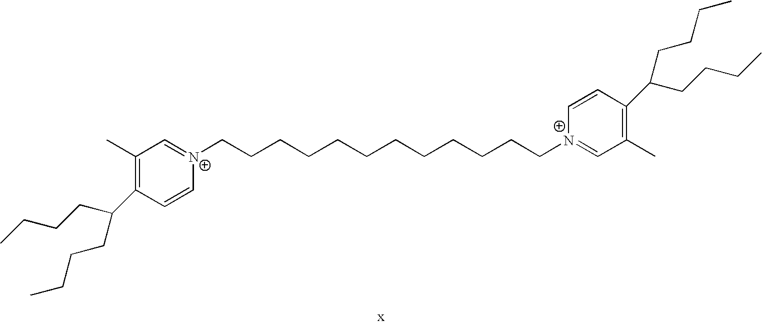

TABLE 1c
Cytotoxicity of bis-(alkylpyridinium)alkane salts

| | | | MIC (μM) | |
|---|---|---|---|---|
| | | | C. | C. |
| | | | neoformans | albicans |
| | IC₅₀ (μM) | | ATCC | ATCC |
| Compounds | A 549^{a)} | MDCK^{b)} | 90112 | 10231 |
| 1,12-bis(4- | 230 | 150 | 2.75 | 2.75 |
| Pentylpyridinium)- | | | | |
| dodecane | | | | |
| 1,12-bis(2- | >350 | 300 | 11 | 2.75 |
| Pentylpyridinium)- | | | | |
| dodecane | | | | |
| 1,12-bis(4- | >350 | >350 | 5.5 | 5.5 |
| Isopentylpyridinium)- | | | | |
| dodecane | | | | |
| 1,12-bis[4- | >350 | >350 | n.d | 2.75 |
| Pentene(1)- | | | | |
| pyridinium]dodecane | | | | |
| 1,12-bis(3,4-Dipentyl- | 40 | 55 | 2.75 | 2.75 |
| pyridinium)dodecane | | | | |
| 1,12-bis[3-Methyl,4- | 10 | 10 | 2.75 | 2.75 |
| pentyl(2- | | | | |
| butyl)pyridinium]dodecane | | | | |
| 12-(4- | 20 | 40 | 2.75 | 5.5 |
| Pentylpyridinium)- | | | | |
| dodecabromide * | | | | |

* monoalkylpyridinium alkyl salt included for comparison.

TABLE 2
In vitro antifungal activity of 1,12-bis(4-pentylpyridinium)
dodecane in comparison to Amphotericin B

| | 1,12-bis(4- | | Amphotericin | |
|---|---|---|---|---|
| | pentylpyridinium)dodecane | | B | |
| | (FW: 537.0) | | (FW: 924.1) | |
| Fungi | μg/ml | μM | μg/ml | μM |
| Aspergillus terreus | 0.7 | 1.2 | 1.0 | 1.1 |
| ATCC 03-232-378 | | | | |
| Aspergillus flavus | 13.4 | 22 | 0.50 | 0.54 |
| ATCC 204 304 | | | | |
| Scedosporium prolificans | 0.75 | 1.4 | 4.0 | 4.3 |
| 1-003-040 | | | | |
| Scedosporium apiospermum | 1.5 | 2.8 | 0.50 | 0.54 |
| 1-003-056 | | | | |
| Fusarium solani | 47 | 88 | 0.25 | 0.27 |
| 04-132-4207 | | | | |
| Cryptococcus neoformans | 1.2 | 2.2 | 0.50 | 0.54 |
| ATCC 90112 | | | | |
| Candida albicans | 0.6 | 1.2 | 0.50 | 0.54 |
| ATCC 10231 | | | | |

TABLE 2a
In-vitro activity of 1,12-bis(4-Pentylpyridinium)dodecane (PYR)
in Comparison with the Commercially Available Antifungal
Drug Itraconazole (ITC) against nine Dermatophyte Species

| | | | | MIC | |
|---|---|---|---|---|---|
| Species (No. of isolates | | | | (μg/ml) | |
| tested) | Compound | Range | GM^{a)} | MIC₅₀^{b)} | MIC₉₀^{c)} |
| Trichophyton rubrum | PYR | 4-16 | 10.41 | 8 | 16 |
| (20) | ITC | 0.25-4 | 1.955 | 2 | 4 |
| Trichophyton mentagrophytes | PYR | 2-32 | 9.007 | 8 | 16 |
| (18) | ITC | 0.125-4 | 1.682 | 2 | 4 |
| Trichophyton tonsurans | PYR | 1-8 | 2.802 | 2 | 8 |
| (16) | ITC | 0.25-4 | 1.645 | 2 | 4 |
| Trichophyton soudanense | PYR | 8-16 | 14.54 | | |
| (3) | ITC | 1-4 | 3.420 | | |
| Trichophyton violaceum | PYR | 1-32 | 8.963 | | |
| (3) | ITC | 2-8 | 3.302 | | |
| Epidermophyton floccosum | PYR | 2-16 | 7.635 | | |
| (5) | ITC | 0.063-0.5 | 0.226 | | |
| Microsporum canis | PYR | 1-4 | 2.954 | | |
| (5) | ITC | 0.25-8 | 1.230 | | |
| Microsporum gypseum | PYR | 1-4 | 2.408 | | |
| (5) | ITC | 1-8 | 3.680 | | |
| Microsporum cookie | PYR | 0.5-2 | 1.061 | | |
| (2) | ITC | 4-8 | 6 | | |

^{a)}GM, geometric mean of the MICs.
^{b)}MIC at which 50% of the isolates were inhibited.
^{c)}MIC at which 90% of the isolates were inhibited.

TABLE 3
Inhibition^{a)} of Secretory Cryptococcal H99 Phospholipase
B and ppPLA₂ Activites by bis(Alkylpyridinium)alkanes.

| | 1,12- | 1,12-bis(4- | 1,14-bis(4- |
|---|---|---|---|
| | bis(Pyridinium)- | Pentylpyridinium)- | Pentylpyridinium)- |
| | dodecane | dodecane | tetradecane |
| PLB | >250 | 12.5 | 6.5 |
| ppPLA₂ | >250 | >250 | >125 |

^{a)}Concentration (μM) required for 50% inhibition of the enzymes

TABLE 4
Hemolytic activity of selected bis(alkylpyridinium)alkane salts as a function
of concentration (in % of positive control which represents 100% lysis)
R—(CH₂)ₙ—R •2Cl⁻

| | | 350 | 175 | 88 | 44 | 17.5 | 3.5 |
|---|---|---|---|---|---|---|---|
| R | n | μM | μM | μM | μM | μM | μM |
| 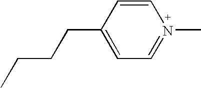 | 12 | 0 | 0 | 0 | 0 | 0 | 0 |
| 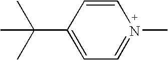 | 12 | 0 | 0 | 0 | 0 | 0 | 0 |
| 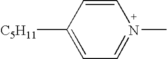 | 8 | 0 | 0 | 0 | 0 | 0 | 0 |
| 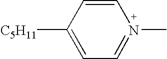 | 10 | 0 | 0 | 0 | 0 | 0 | 0 |
| 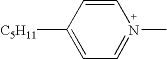 | 12 | 0 | 0 | 0 | 0 | 0 | 0 |
| 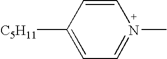 | 14 | 26 | 0 | 0 | 0 | 0 | 0 |
| 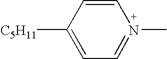 | 16 | 100 | 35 | 5 | 0 | 0 | 0 |
| 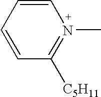 | 12 | 0 | 0 | 0 | 0 | 0 | 0 |
| 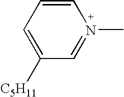 | 12 | 0 | 0 | 0 | 0 | 0 | 0 |
| 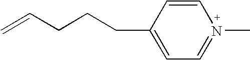 | 12 | 0 | 0 | 0 | 0 | 0 | 0 |
| 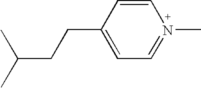 | 12 | 0 | 0 | 0 | 0 | 0 | 0 |
| 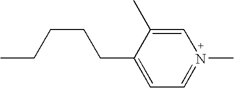 | 12 | 20 | 5 | 0 | 0 | 0 | 0 |
| 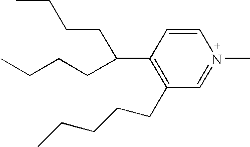 | 12 | 100 | 30 | 15 | 5 | 0 | 0 |
| 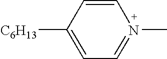 | 12 | 100 | 85 | 5 | 0 | 0 | 0 |
| 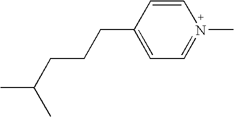 | 12 | 95 | 5 | 0 | 0 | 0 | 0 |
| 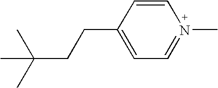 | 12 | 0 | 0 | 0 | 0 | 0 | 0 |
| 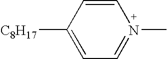 | 12 | 100 | 100 | 95 | 10 | 0 | 0 |
| 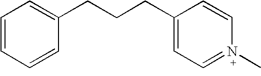 | 12 | 100 | 100 | 80 | 0 | 0 | 0 |
| 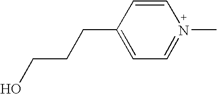 | 12 | 0 | 0 | 0 | 0 | 0 | 0 |
| 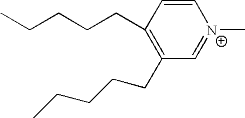 | 12 | 100 | 100 | 100 | 50 | 50 | 0 |
| 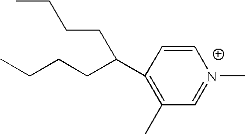 | 12 | 100 | 100 | 100 | 75 | 5 | 0 |
| 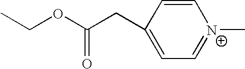 | 12 | 0 | 0 | 0 | 0 | 0 | 0 |

TABLE 5
In vitro antibacterial properties of 1,12-bis(4-pentylpyridinium)
dodecane [FW: 537.0]

MIC

| | | |
|---|---|---|
| Bacteria | μg/ml | μM |

Gram-negative

| | | |
|---|---|---|
| Escherichia coli^{a)} 25922 | 5.9 | 11 |
| Pseudomonas aeruginosa^{b)} 122 | 86 | 160 |

Gram-positive

| | | |
|---|---|---|
| Staphylococcus aureus^{c)} 25923 | 3.0 | 5.5 |
| MRSA^{d)} (methicillin resistant S. aureus) | 1.45 | 2.7 |
| Streptococcus pneumonia^{e)} 49619 | 1.45 | 2.7 |
| VRE^{f)} (Vancomycin resistant Enterococcus) | 5.9 | 11 |

Positive control was amoxicillin (FW: 365.4). MIC obtained was 8-16 μg/ml
^{b)}Positive control was Gentamicin (FW: 470). MIC obtained was 1.0 μg/ml.
^{c)}Positive control was amoxicillin (FW: 365.4). MIC obtained was 0.25-0.50 μg/ml
^{d)}Positive control was vancomycin (FW: 1485). MIC obtained was 1.0 μg/ml
^{e)}Positive control was amoxicillin (FW: 365.4). MIC obtained was 0.06-0.12 μg/ml.
^{f)}Positive control does not exist

1. A method of treating, inhibiting, or preventing an infection in a subject, said method comprising administering to said subject an effective amount of at least one bis-pyridinium compound, wherein said bis-pyridinium compound comprises two aromatic ring structures and wherein:
each of the ring structures comprises a pyridine ring,
the ring structures are linked by a linker group of at least 8 atoms in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group.

2. The method of claim 1 wherein each of the ring structures is, independently, a pyridine ring or a fused pyridine ring.

3. The method of claim 1 wherein the bis-pyridinium compound has structure I, 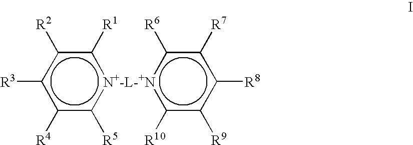
wherein
at least one of R¹ to R¹⁰ is an alkyl group having at least 2 carbon atoms,
none of R¹ to R¹⁰ is —OH, —SH or an amine group, and
L is a linker group which is at least 8 atoms in length.

4. The method of claim 1, wherein the linker group is between 8 and 18 atoms long.

5. The method of claim 1, wherein the main chain of the linker group comprises a hydrocarbon chain.

6. The method of claim 1, wherein no substituent on either ring structure, other than the linker group, has more than 10 carbon atoms in a straight chain.

7. The method of claim 1, wherein the substitution on the two ring structures is the same.

8. The method of claim 1, wherein the bis-pyridinium compound has an MIC against C. neof. ATCC 90112 or against C. albicans ATCC 10231 of less than about 11 micromolar or less than about 10 micrograms per millilitre.

9. The method of claim 1, wherein the infection is a microbial infection, a bacterial infection, a fungal infection, an amoebic infection, a viral infection, a parasitic infection or a helminthic infection.

10. The method of claim 1, wherein the bis-pyridinium compound is administered topically.

11. The method of claim 1, wherein the bis-pyridinium compound is administered systemically.

12. The method of claim 1, wherein the patient is an animal or a plant.

13. A method of killing an organism, or of inhibiting or preventing growth of the organism, comprising exposing said organism to an effective amount of at least one bis-pyridinium compound, wherein said bis-pyridinium compound comprises two aromatic ring structures and wherein:
each of the ring structures comprises a pyridine ring,
the ring structures are linked by a linker group of at least 8 atoms in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group.

14. The method of claim 13 wherein the organism is a bacterium, a fungus, an amoeba, a parasite, a virus, a helminth, a mould or a nematode.

15. A biocidal bis-pyridinium compound comprising two aromatic ring structures wherein:
each of the ring structures comprises a pyridine ring,
the ring structures are linked by a linker group of at least 8 atoms in length, said linker group being attached to the nitrogen atoms of the pyridine rings,
at least one substituent on at least one of the ring structures is an alkyl group having at least 2 carbon atoms, and
no substituent on either of the ring structures is —OH, —SH or an amine group;
wherein said compound is not one of the following compounds: 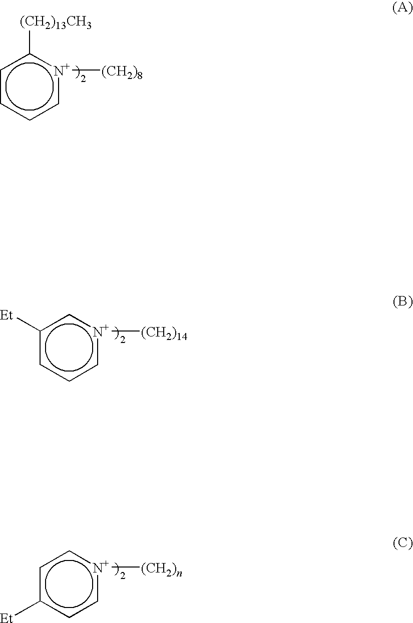
wherein n=8, 10 or 12 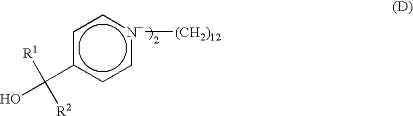
wherein either R¹═R²=Et or R¹=Me and R²=Me, Et, Pr, Bu, allyl or 4-butenyl 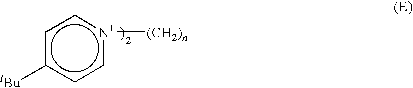
wherein n=8, 9 or 10 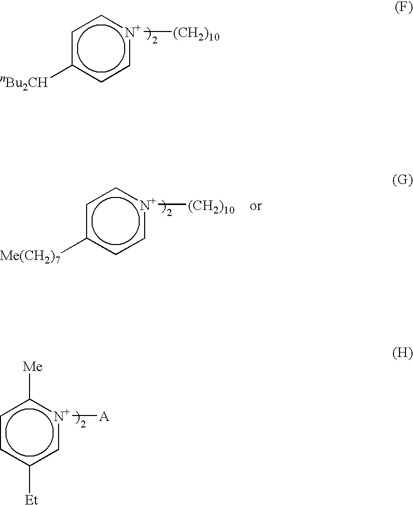
wherein A=(CH₂) or ((CH₂)₂O)₈(CH₂)₂,
said compound having an MIC against C. neof. ATCC 90112 or against C. albicans ATCC 10231 of less than about 11 micromolar or less than about 10 micrograms per millilitre.

16. The bis-pyridinium compound of claim 15 wherein the linker group is between 10 and 18 atoms long.

17. The bis-pyridinium compound of claim 15 wherein the linker group comprises a hydrocarbon chain.

18. The bis-pyridinium compound of claim 15, wherein no substituent on either ring structure, other than the linker group, has more than 10 carbon atoms in a straight chain.

19. The method of claim 1,
wherein said compound is not one of the following compounds: 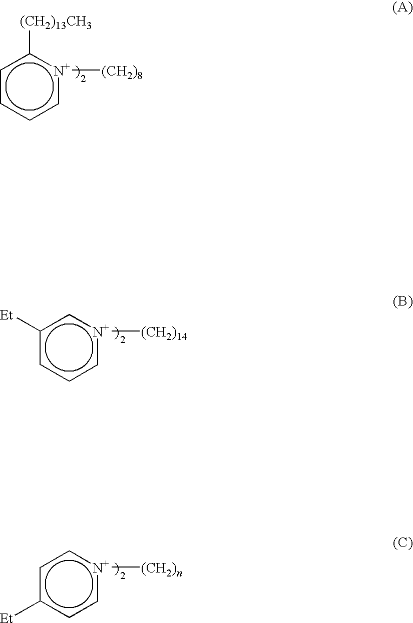
wherein n=8, 10 or 12 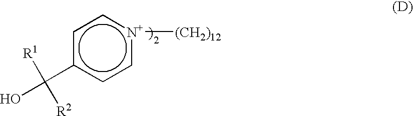
wherein either R¹═R²=Et or R¹=Me and R²=Me, Et, Pr, Bu, allyl or 4-butenyl 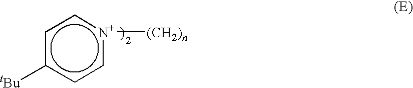
wherein n=8, 9 or 10 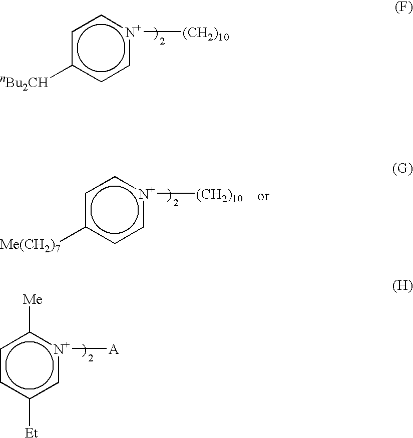
wherein A=(CH₂) or ((CH₂)₂O)₈(CH₂)₂,
said compound having an MIC against C. neof ATCC 90112 or against C. albicans ATCC 10231 of less than about 11 micromolar or less than about 10 micrograms per millilitre.

20. The compound of claim 15 further comprising at least one acceptable adjuvant or carrier.

21. The method of claim 13,
wherein the compound is not one of the following compounds: 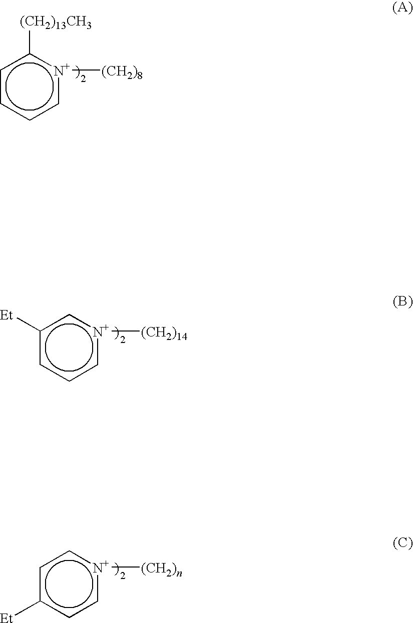
wherein n=8, 10 or 12 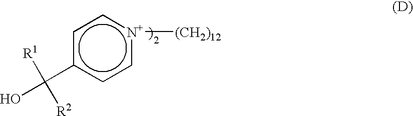
wherein either R¹═R²=Et or R¹=Me and R²=Me, Et, Pr, Bu, allyl or 4-butenyl 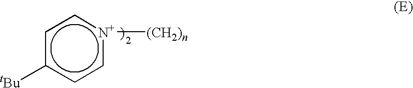
wherein n=8 9 or 10 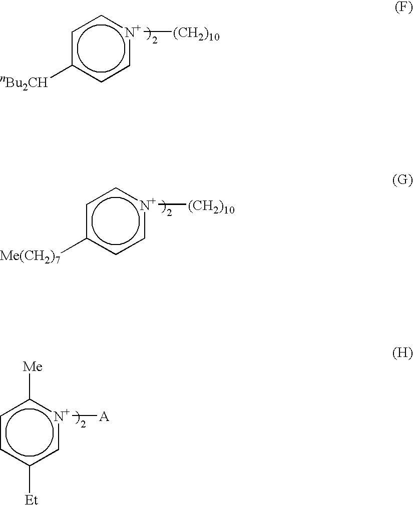
or
wherein A=(CH₂) or ((CH₂)₂O)₈(CH₂)₂.
